(19) United States
(12) Patent Application Publication
(10) Pub. No.: US 2024/0166982 A1
(43) Pub. Date: May 23, 2024

(54) FUNCTIONALLY GRADED BIOMATERIAL STRUCTURES FOR PROGRAMMABLE TISSUE AND ORGAN BIOFABRICATION

(71) Applicant: THE TRUSTEES OF THE STEVENS INSTITUTE OF TECHNOLOGY, Hoboken, NJ (US)

(72) Inventors: Dilhan KALYON, Teaneck, NJ (US); Filippos TOURLOMOUSIS, Cambridge, MA (US); Robert CHANG, Wayne, NJ (US)

(73) Assignee: THE TRUSTEES OF THE STEVENS INSTITUTE OF TECHNOLOGY, Hoboken, NJ (US)

(21) Appl. No.: 18/550,106

(22) PCT Filed: Mar. 21, 2022
(86) PCT No.: PCT/US2022/021200

§ 371 Date: Sep. 11, 2023

Related U.S. Application Data
(60) Provisional application No. 63/163,703, filed on Mar. 19, 2021.

(51) Int. Cl. C12M 1/12; B33Y 10/00; B33Y 50/00; B33Y 80/00; C12M 3/00

(52) U.S. Cl. CPC C12M 25/14; B33Y 50/00; B33Y 80/00; C12M 21/08; B33Y 10/00

(57) ABSTRACT

A biomaterial structure for proliferation of stem cells includes at least one lattice sub-structure, and a structural gradient in which one or more geometrical features of the biomaterial structure varies along at least one dimension of the biomaterial structure in three-dimensional space. In some embodiments, the structural gradient is accomplished by the first and second lattice sub-structures having at least one different geometrical parameter.

### BACKGROUND

Stem cells are unspecialized cells that can replicate themselves through cell division multiple times (e.g., proliferate) while remaining unspecialized, or can differentiate into tissue- or organ-specific cells like nerve, blood, fat and heart muscle cells. Stem cells are differentiated into different cell types during early life and growth, as well as for the repair and replacement of cells of diseased, damaged or worn-out tissues.

There are two kinds of stem cells: 1) embryonic stem cells (from human embryos) which can remain undifferentiated for a year or more; and 2) adult stem cells, (referred to as somatic cells, i.e., the cells of the body and not those of the sperm, eggs, germs). Adult stem cells are found in the stem cell niches of many organs and tissues, and generally remain in their niches undifferentiated for years, proliferating and differentiating only when they are prompted primarily by tissue injury or disease. For example, bone marrow contains hematopoietic stem cells that differentiate to form all types of blood cells (e.g., B lymphocytes, T lymphocytes, red blood cells, neutrophils, natural killer cells, eosinophils, basophils, macrophages, and monocytes), and bone marrow stromal stem cells (mesenchymal stem cells or skeletal cells) that differentiate into bone (osteoblasts and osteocytes), cartilage (chondrocytes), fat (adipocytes), and stromal cells that support blood formation. Stem cells found in the brain can differentiate into astrocytes, oligodendrocytes and neurons. Some specialized human adult cells can be converted also into pluripotent stem cells upon genetic reprogramming. The adult stem cells can potentially be directed to differentiate (or, “transdifferentiate”) into unrelated cell types (e.g., skin stem cells directed to differentiate into blood cells, or blood-forming cells directed to differentiate into cardiac muscle cells).

Adult stem cells can be removed from the body of the patient or from another donor, from the amniotic fluid, or can be obtained from the directed differentiation of embryonic stem cells and induced pluripotent cells (iPS cells). Adult stem cells can be isolated from the body in different ways, depending on the tissue. Blood stem cells, for example, can be taken from a donor's bone marrow, from blood in the umbilical cord when a baby is born, or from a person's circulating blood. Mesenchymal stem cells (which can make bone, cartilage, fat, fibrous connective tissue, and cells that support the formation of blood) can also be isolated from bone marrow. Neural stem cells (which form the brain's three major cell types) can be isolated from the brain and spinal cord. Cardiac stem cells can be harvested from the heart.

Amniotic fluid contains fetal cells (i.e., arising from the fetus) including mesenchymal stem cells of the fetus. The amniotic fluid is occasionally drawn during pregnancy, typically for amniocentesis, i.e., to test for chromosomal defects. This withdrawn amniotic fluid can be used to isolate fetal mesenchymal stem cells.

Embryonic stem cells and induced pluripotent cells (iPS cells) can be used to produce various kinds of adult stem cells.

Laboratory investigations using human stem cells can be employed as model systems to study development of diseases and birth defects and to screen new drugs. New medications may be tested for safety on differentiated cells generated from human pluripotent cell lines and on disease models. For example, cancer cell lines can be used to screen potential anti-tumor drugs. To screen drugs effectively, the screening conditions must be identical when comparing different drugs. When using stem cells, the differentiation of stem cells into the specific cell types on which drugs will be tested needs to be controlled. However, knowledge of the signals controlling differentiation is lacking, and industrially it is currently not possible to mimic cell culturing conditions in such a precise way so that pure populations of differentiated cells are obtained and used for each drug being tested.

Typically, stem cells are transferred into culture dishes that contain nutrients, (i.e., culture media). When the cells divide and spread (i.e., proliferate) to cover the surface of the culture dish, they are removed and plated into fresh culture dishes, with each cycle of proliferation referred to as a “passage.”

For the identification of adult stem cells, the cells in living tissue are labeled with molecular markers followed by the determination of the specialized cell types they generate. To be useful, the harvested adult stem cells should be able to proliferate to generate a line of genetically identical cells (i.e., “retain their stemness”) which can then be directed to differentiate into other cell types so that they can be transplanted for tissue repair.

Stem cells need to be characterized on a regular basis to assure that they remain undifferentiated. This can be accomplished using different methods including microscopy, the determination of transcription factors (including Nanog and Oct4), the characterization of cell surface markers, or injecting the cells into animals with suppressed immune systems to follow their differentiation in vivo. To determine if the cells are retaining their pluripotency, the cells can be manipulated to differentiate (i.e., directed to differentiate via changes in the composition of the culture medium, modification of the surface of the culture dish, and modification of the cells by inserting specific genes). Furthermore, the cells can be directed to differentiate spontaneously by allowing them to clump together to form embryoid bodies.

If the stem cells proliferate in culture for more than six months without differentiation (pluripotent stem cells) they form a stem cell line that can be frozen, shipped, thawed and used for research or transplantation. However, it may also be desirable not only to proliferate, but also to direct the differentiation of stem cells so that they can be transplanted in the body to treat injured or diseased tissues.

Indeed, one potential application of human stem cells is the generation of cells and tissues that could be used for cell-based therapies. Today, donated organs and tissues are typically used to replace damaged or diseased tissue. But the availability of such donated organs and tissue is very limited. Stem cells directed to differentiate into specific cell types offer the possibility of a renewable source of replacement cells and tissues to treat diseases including macular degeneration, spinal cord injury, stroke, burns, heart disease, type 1 diabetes, osteoarthritis, rheumatoid arthritis, etc.

One of the challenges of donated organs and tissues is the likelihood of rejection after transplantation. This is a major problem associated with cell based therapies which are non-autologous (i.e., the donor and the patient are not the same). Overcoming immune rejection requires the continuous administration of immunosuppressive drugs, which carry significant side effects. However, the ability to use adult stem cells and tissues derived from the patient's own adult stem cells would significantly lessen the likelihood of rejection by the patient's immune system. For example, fetal mesenchymal stem cells harvested from the amniotic fluid could be used to grow new tissues for babies with birth defects, such as congenital diaphragmatic hernia. These tissues would match the baby genetically, would therefore likely not be rejected by the immune system, and could be implanted either in utero or after the baby is born.

Unfortunately, adult stem cells are not numerous in mature tissues and it is therefore a challenge to isolate and harvest them. Also, unlike embryonic stem cells, the ability of adult stem cells to divide outside of the body is limited. Therefore, it is a challenge to find ways to enable the proliferation of large numbers of adult stem cells in culture. It is also a challenge to retain stem cells in an undifferentiated state outside of the body until directed to differentiate. It is therefore also a challenge to direct adult stem cells to differentiate into specific cell types so that they can be used for regenerative medicine. Thus, the ability to do so would mark a significant achievement in the advancement of regenerative medicine and therapy.

### SUMMARY

According to embodiments of the present disclosure, a biomaterial structure for proliferation of stem cells include at least one lattice sub-structure, and has a structural gradient in which one or more geometrical features of the biomaterial structure varies along at least one dimension of the biomaterial structure in three-dimensional space. The at least one lattice sub-structure may include at least first and second lattice sub-structures. And the structural gradient of the biomaterial structure may be accomplished by the first and second lattice sub-structures having at least one different geometrical parameter.

In some embodiments, the at least one lattice sub-structure, the first and second lattice sub-structures, or the biomaterial structure may be constructed of filaments having a diameter of about 10 micrometers to about 100 micrometers.

According to embodiments, the at least one lattice sub-structure, the at least first and second lattice sub-structures, or the biomaterial structure may include a first plurality of lattice sub-structures assembled into a first biomaterial substrate module; and a second plurality of lattice sub-structures assembled into a second biomaterial substrate module, and the first and second biomaterial substrate modules may be assembled into a multi-module biomaterial substrate. At least two of the first plurality of lattice sub-structures of the first biomaterial substrate module may have at least one different geometrical parameter. Each of the second plurality of lattice sub-structures of the second biomaterial substrate module may have identical geometrical parameters. In some embodiments, each of the first plurality of lattice sub-structures of the first biomaterial substrate module may have identical geometrical parameters, each of the second plurality of lattice sub-structures of the second biomaterial substrate module may have identical geometrical parameters, and the first plurality of lattice sub-structures of the first biomaterial substrate module and the second plurality of lattice sub-structures of the second biomaterial substrate module have at least one different geometrical parameter.

In some embodiments, the at least one lattice substructure, the at least first and second lattice sub-structures, or the biomaterial structure may include a first plurality of lattice sub-structures assembled into a first biomaterial substrate module, and the second lattice sub-structure, and the first biomaterial substrate module and the second lattice sub-structure may be assembled into a multi-module biomaterial substrate. At least two of the first plurality of lattice sub-structures of the first biomaterial substrate module may have at least one different geometrical parameter. Each of the first plurality of lattice sub-structures of the first biomaterial substrate module may have identical geometrical parameters.

According to some embodiments, a method of making a biomaterial structure designed to grow a specified tissue formation or organ structure mimicking a native tissue formation or native organ structure includes generating a digital model of the biomaterial structure from a database correlating predicted cell differentiation types or predicted long term tissue structures with lattice sub-structures having specified geometric parameters. The digital model includes: at least one lattice sub-structure having a structural gradient identified by the database as needed to form each tissue type needed to mimic the native tissue formation or native organ structure, the structural gradient being such that one or more geometrical features of the biomaterial structure varies along at least one dimension of the biomaterial structure in three-dimensional space; and/or a combination of lattice sub-structures identified by the database as needed to form each tissue type needed to mimic the native tissue formation or native organ structure. The method further includes constructing or printing the biomaterial structure using the digital model.

In some embodiments, the digital model may include the at least one lattice sub-structure having the structural gradient, a combination of different lattice sub-structures, a combination of different biomaterial substrate modules, or a combination of at least one lattice sub-structure and at least one biomaterial substrate module.

According to some embodiments, the constructing or printing the biomaterial structure using the digital model comprises 3D printing the biomaterial structure using the digital model as an instruction or template. In some embodiments, the constructing or printing the biomaterial structure using the digital model comprises manually connecting or assembling the biomaterial structure using the digital model as an instruction or template.

In some embodiments of the present disclosure, a method of tuning early single cell shape on a biomaterial structure includes varying the physical characteristics of the biomaterial structure in order to guide long term tissue function, where early single cell shape is the single cell shape formed 24 hrs after the cell has been seeded. According to some embodiments, varying the physical characteristics of the biomaterial structure comprises imparting the biomaterial structure with a structural gradient in which one or more geometrical features of the biomaterial structure varies along at least one dimension of the biomaterial structure in three-dimensional space.

In some embodiments, the biomaterial structure comprises at least one lattice sub-structure, and the varying the physical characteristics of the biomaterial structure comprises imparting the at least one lattice sub-structure with a structural gradient in which one or more geometrical features of the lattice substructure varies along at least one dimension of in three-dimensional space. In some embodiments, the at least one lattice sub-structure or the biomaterial structure may include at least first and second lattice substructures. The structural gradient of the biomaterial structure may be accomplished by the first and second lattice sub-structures having at least one different geometrical parameter. In some embodiments, the at least one lattice sub-structure, the first and second lattice sub-structures or the biomaterial structure may be constructed of filaments having a diameter of about 10 micrometers to about 100 micrometers.

In some embodiments, the at least one lattice sub-structure, the at least first and second lattice sub-structures, or the biomaterial structure may include a first plurality of lattice sub-structures assembled into a first biomaterial substrate module, and a second plurality of lattice sub-structures assembled into a second biomaterial substrate module, and the first and second biomaterial substrate modules may be assembled into a multi-module biomaterial substrate. At least two of the first plurality of lattice sub-structures of the first biomaterial substrate module may have at least one different geometrical parameter. Each of the second plurality of lattice sub-structures of the second biomaterial substrate module may have identical geometrical parameters.

In some embodiments, each of the first plurality of lattice sub-structures of the first biomaterial substrate module may have identical geometrical parameters, each of the second plurality of lattice sub-structure of the second biomaterial substrate module may have identical geometrical parameters, and the first plurality of lattice sub-structures of the first biomaterial substrate module and the second plurality of lattice sub-structures of the second biomaterial substrate module may have at least one different geometrical parameter.

According to some embodiments, each of the at least one lattice substructure, the at least first and second lattice sub-structures, or the biomaterial structure may include a first plurality of lattice sub-structures assembled into a first biomaterial substrate module, and the second lattice sub-structure, and the first biomaterial substrate module and the second lattice sub-structure may be assembled into a multi-module biomaterial substrate.

In some embodiments, at least two of the first plurality of lattice sub-structures of the first biomaterial substrate module may have at least one different geometrical parameter. And in some embodiments, each of the first plurality of lattice sub-structures of the first biomaterial substrate module may have identical geometrical parameters.

### BRIEF DESCRIPTION OF THE DRAWINGS

These and other features and advantages of the present disclosure will be better understood with reference to the detailed description when considered in conjunction with the accompanying drawings, in which:

FIG. 1 is a schematic diagram depicting the construction of a database correlating lattice sub-structure geometries to cell phenotypes and tissue types according to embodiments of the present disclosure;

FIG. 2A is a schematic illustrating the assembly or connection of three different lattice substructures to form a heterogeneous biomaterial substrate module according to embodiments of the present disclosure;

FIGS. 2B-2D are schematic illustrations of different biomaterial substrate modules constructed from the lattice sub-structures depicted in FIG. 2A according to embodiments of the present disclosure.

FIG. 3A is a schematic illustrating the assembly or connection of six different lattice sub-structures to form a multi-module biomaterial substrate according to embodiments of the present disclosure;

FIG. 3B is a schematic illustration of a multi-module biomaterial substrate constructed from the lattice sub-structures of FIG. 3A according to embodiments of the present disclosure.

FIG. 4 is a schematic depicting the constructions of an “interface tissue” from a heterogeneous biomaterial substrate module according to embodiments of the present disclosure;

FIG. 5 is an illustration of a complex native tissue formation, which could potentially be used as a model for constructing a multi-module biomaterial substrate (e.g., using the lattice sub-structures and biomaterial substrate modules according to embodiments of the present disclosure) for growth or formation of tissue mimicking the native tissue formation according to embodiments of the present disclosure;

FIG. 6A is a schematic diagram illustrating a melt electrowriting apparatus according to embodiments of the present disclosure;

FIG. 6B is a schematic diagram depicting a custom built manufacturing system, according to embodiments of the present disclosure;

FIG. 7 is a screen-capture image of a thermogram and associated data display depicting the custom built manufacturing system of FIG. 6;

FIG. 8 is a reproduction of a photographic image of a portion of the custom built manufacturing system of FIG. 6;

FIG. 9 is a graph of the operating centigrade temperature of the custom built manufacturing system of FIG. 6 as a function of the distance between the tip and the collector;

FIG. 10 is a schematic illustrating a proposed heating element according to embodiments of the present disclosure;

FIG. 11 is a schematic illustrating the key heat transfer mechanisms in the polymer melt supply and free-flow regime according to embodiments of the present disclosure;

FIGS. 12-16 are a set of reproductions of photographic images showing scaffolds fabricated from poly(caprolactone) (“PCL”) melts by a method according to embodiments of the present disclosure, the scaffolds having different configurations according to embodiments of the present disclosure, and the scaffolds of FIGS. 14-15 specifically being woven scaffolds;

FIGS. 17 and 18 are reproductions of photographic images, and respective enlarged sub-figures (FIGS. 17A and 18A), showing fibrous scaffolds fabricated from PCL melts by a method according to embodiments of the present disclosure, the scaffolds having a woven configuration with different porous microarchitectures. The FIG. 21 scaffold has a MEW I 0-90° configuration, and the scaffold D has a MEW I 0-45° configuration;

FIG. 19 is a schematic diagram providing an overview of a cell classification method according to embodiments of the present disclosure;

FIG. 20 is a flow diagram of a feature extraction algorithm in accordance with embodiments of the present disclosure;

FIGS. 21-24 are a group of reproductions of photographic immunofluorescence images showing cellular structures observed during stem cell expansion by a method according to embodiments of the present disclosure, wherein FIG. 21 is a grayscale multi-channel maximum projection image obtained by combining three different single channel maximum projections, the single channel maximum projections obtained by processing Z-stack raw images, wherein the red channel is associated with the cytoskeleton, the blue channel is associated with the nucleus, and the green channel is associated with vinculin. FIG. 22 is a grayscale maximum projection of the red channel cell body image overlaid with the contour of the segmented cell body, FIG. 23 is a grayscale maximum projection of the blue channel image overlaid with the contour of the segmented nucleus, and FIG. 24 is a grayscale maximum projection of the green channel image overlaid with the contour of the segmented focal adhesions (scale bar: 20 μm);

FIGS. 25-33 are graphical illustrations of examples of a feature extraction procedure using single-cell automated bioimage analysis of immunofluorescent images by a method according to embodiments of the present disclosure, providing a demonstration of the performance of an automated image processing algorithmic workflow according to embodiments of the present disclosure that uses a representative cell cultured in 3-D microscale fibrous scaffold. FIGS. 25-33 illustrate an algorithmic procedure according to embodiments of the present disclosure that allows the development of critical cellular and subcellular focal adhesion morphometric and distribution metrics that are useful for the training and application of the developed classification method to various cell types according to embodiments of the present disclosure;

FIGS. 34-39 present graphical examples (FIGS. 34, 36 and 38) and confusion matrices (FIGS. 35, 37 and 39) illustrating the use of the classification methodology according to embodiments of the present disclosure to different scaffold geometries, and the confinement states of stem cells within the scaffolds during expansion, the graphical examples and confusion matrices documenting changes in cellular and subcellular adhesion proteins for the different geometries (for all cells under analysis >100), and demonstrating that the novel 3-D substrate architectures according to embodiments of the present disclosure induce uniform and geometry dependent cell shapes and resulting phenotypes while, in contrast, the control stem cell cultures on flat surfaces or non-woven 2-D meshes with randomly oriented fibers induce heterogeneous cell shapes, presumably inducing phenotype heterogeneities; and

FIG. 40 is a schematic diagram of a concept for industrial exploitation of the classification method according to embodiments of the present disclosure, further including feedback and feedforward control methodologies for the programmable expansion and harvesting of stem cells having phenotypes that are targeted and realized by a method according to embodiments of the present disclosure.

### DETAILED DESCRIPTION

A key challenge in tissue engineering and regenerative medicine research is how to direct the differentiation of stem cells toward specific fates by engineering in vitro models with cell-instructive microenvironments. Specific ligand-receptor interactions of growth factors and matrix molecules are important for regulating cells. Various topographical patterning techniques have been employed to pattern bioactive molecules on two-dimensional flat surfaces. In addition to that, microfluidics technologies have been employed to apply dynamic chemical gradients, through a process known as chemotaxis. Furthermore, the physical properties of the local microenvironment (such as the elasticity of the matrix microenvironment) can also play key roles in determining cellular function and fate, through a process known as durotaxis. By changing the stiffness of the substrate, human mesenchymal stem cells could be directed along neuronal, muscular, or bone lineages. To this end, 2D in vitro models with tightly controlled chemotactic and durotactic microenvironments can be used as tools for investigating the control of stem cell differentiation. The design of physical signal gradients (durotactic-like) within three-dimensional (3D) biomaterial constructs has tremendous potential for clinical applications considering the strict regulatory pathway around biologics and drugs, whose resultant therapeutic/biological functionality stems from chemical signals.

It is possible to fabricate tissue engineering substrates containing systematic gradients in the distributions of biological stimulators. For example, as discussed in C. Erisken, D. M. Kalyon and H. Wang, “Functionally and continuously graded electrospun polycaprolactone and β-tricalcium phosphate nanocomposites for interface tissue engineering applications”, Biomaterials 29, 4065-4073 (2008), the entire contents of which are incorporated herein by reference as noted below, using a hybrid twin screw extrusion and electrospinning method, the concentration distributions of two bioactive agents (e.g., insulin and β-glycerol phosphate) can be varied concomitantly (e.g., one increasing; the other decreasing monotonically) in between the two sides of a nanofibrous substrate to generate gradients of, e.g., insulin (a stimulator of chondrogenic differentiation), and e.g., β-glycerophosphate (β-GP) (for mineralization). C. Erisken, D. Kalyon, C. Ornek, H. Wang and J. Xu, “Osteochondral tissue formation through adipose-derived stem cell differentiation using biomimetic tissue scaffolds with graded stimulator concentrations”, Tissue Engineering: Part A, 17, 9, 1239-1252 (2011), the entire contents of which are incorporated herein by reference, as noted below. When graded poly(caprolactone) meshes are seeded with human adipose-derived stromal cells and cultured over 8 weeks, the resulting tissue constructs have revealed the selective differentiation of human adipose-derived stromal cells toward chondrogenic lineage and mineralization as functions of position as a result of the corresponding concentrations of insulin and β-GP. Chondrogenic differentiation of the stem cells increased at insulin-rich locations, and mineralization increased at β-GP-rich locations. It should be noted that the gradations were generated using different bioactive molecules, the concentrations of which were varied systematically. Furthermore, the substrates were fibrous meshes with random “non-regular” structures.

It is also possible to uniformly guide the stemness of mesenchymal stem cells from the bone marrow through single cell shape manipulation on precision manufactured 3D biomaterial scaffolds with lattice microarchitecture. U.S. patent application Ser. No. 15/998,685 (now U.S. Pat. No. 11,078,459, issued Aug. 3, 2021), titled “Integrated methods for precision manufacturing of tissue engineering scaffolds,” to Tourlomousis et al. (assigned to the same Assignee as the present application), the entire content of which is incorporated herein by reference, as noted below. As discussed in Tourlomousis, the uniformity and morphometric features of the acquired cell shapes is causally related to the uniformity and scale of the geometrical features that specify the lattice microarchitecture of the underlying cell culture platforms. Nonlimiting examples of these geometrical features include the filament specifications, along with the pore specifications as defined by the inter-filament distance and filament orientation parameters. And Tourlomousis discloses methods for the precise control of the porous microarchitecture of a 3-D scaffold with cellular-relevant geometrical feature sizes, thereby providing control of the shapes and the phenotypes of the expanded stem cells. Those methods combine melt electrospinning and additive manufacturing, and can be used to fabricate scaffold meshes of unmatched geometrical fidelity and precision, including fibrous architectures with consistent fiber diameters, orientations, alignment, and interconnectivities.

According to some embodiments of the present invention, stem cell shapes and tissue formations (including, e.g., organ structures) may be manipulated, predicted or controlled by tailoring the geometries of the 3-D substrates on which the stem cells are grown. This may provide a shape-driven pathway to control the phenotype of the stem cells (e.g., cell morphology and cell-specific function). Lattice sub-structures with geometric features (e.g., filament and/or pore specifications) having the same size scale as that of a single typical eukaryotic cell (e.g., 10-100 microns, or 10-20 microns) enables the cells to form directed adhesions to the lattice sub-structures, which allows control of the resulting cell shapes by adjusting (or tailoring) the geometries of the lattice sub-structure. According to embodiments of the present disclosure, these lattice sub-structures can then be combined to form more complex scaffolds (or biomaterial structures) with graded geometries across different directions in three-dimensional space, enabling formation of different tissue types and tissue formations (including, e.g., organ structures). These graded geometries are also referred to herein as “structural gradients,” and are distinct and novel compared to existing, conventional durotactic-like stiffness gradients. Indeed, the structural gradients according to embodiments of the present disclosure regulate biological functionality through user-designed geometrical features that are implemented robotically during a printing or manufacturing process. In contrast, conventional stiffness gradients regulate cellular response using the intrinsic material property of stiffness, which is a result of the molecular structure of the raw material and can be tuned by including either fillers, or molecules that induce light- and/or chemical-cross-linking pathways. In some embodiments, for example, the structural gradients according to embodiments of the present disclosure are imparted by modifying or adjusting the physical, geometrical features of the filaments (e.g., filament diameter) and their spacing relative to one other (e.g., inter-filament distance and filament orientation), which thereby adjusts or modifies the porous microarchitecture of the lattice substructure (or of the specified region of the lattice substructure). And by adjusting these geometrical features in different areas (or regions) of the lattice substructure (or by connecting multiple different lattice substructures together), a structural gradient may be created in which one or more of the geometrical features varies along at least one dimension in three-dimensional space.

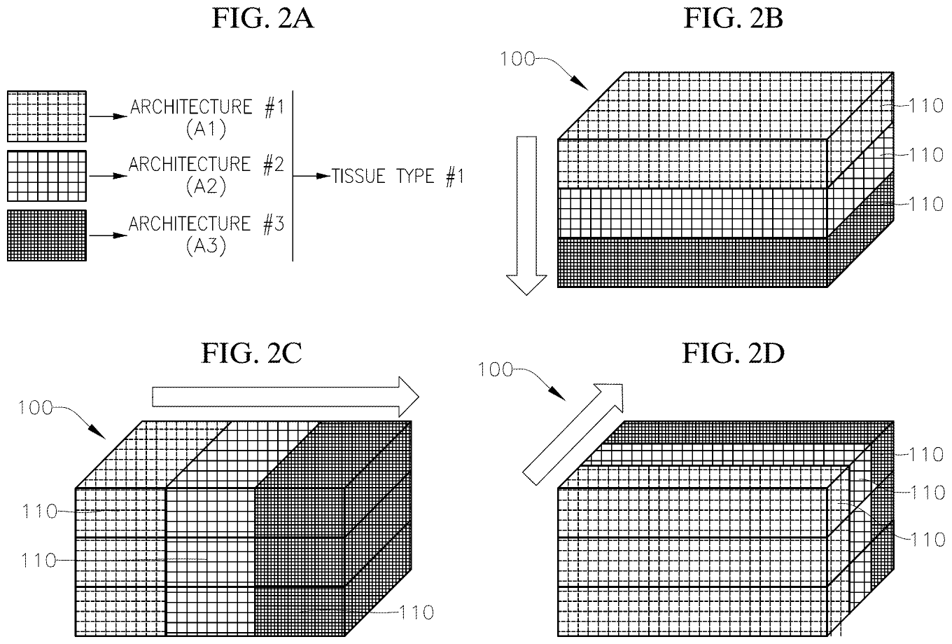

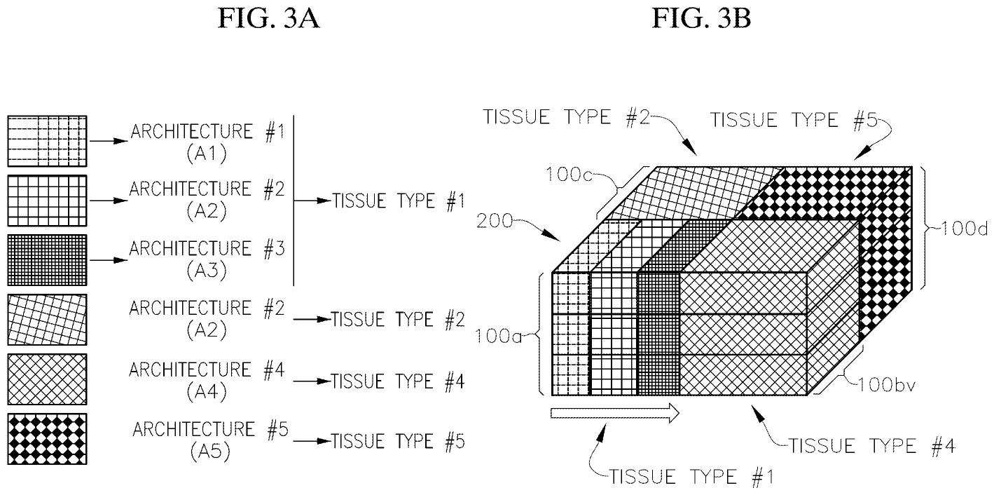

According to embodiments of the present disclosure, and referring to FIGS. 2B-2D and 3B, heterogeneous biomaterial substrate modules 100 have functionally graded geometries that can be tailored to achieve a desired cell shape and/or long-term tissue formation. These functionally graded heterogeneous biomaterial substrates 100 can be constructed using a programmable multimodal tissue fabrication database, and can include multiple different lattice microarchitectures (also referred to herein, interchangeably, as “lattice sub-structures”) 110. For example, a heterogeneous biomaterial substrate module 100 according to embodiments of the present disclosure may include a plurality of lattice sub-structures 110 connected together and having at least one different geometrical lattice parameter (e.g., fiber diameter, inter-fiber spacing and/or inter-layer angle).

Additionally, in some embodiments, a plurality of heterogeneous biomaterial substrate modules 100 can be combined (or connected) to form a multi-module biomaterial substrate 200 which can be used to form multiple tissue types using a single scaffold (or structure). In some embodiments, however, the multi-module biomaterial substrate 200 may include a plurality of different homogeneous biomaterial substrate modules (shown as 100b, c and d in FIG. 3B, for example), or any combination of heterogeneous and homogeneous biomaterial modules. As discussed elsewhere herein, the combination of different homogenous modules or the combination of homogenous modules with at least one heterogeneous module (as shown in FIG. 3B, for example) can yield complex tissue formations (including, e.g., organ structures) having both a structural gradient and multiple tissue types. As used herein, the term “heterogeneous biomaterial substrate module” refers to biomaterial substrate modules that include a plurality of lattice sub-structures, at least two of which have different lattice geometries (as that term is defined herein). An example of such a heterogeneous biomaterial substrate module is shown as 100a in FIG. 3B. Conversely, the term “homogeneous biomaterial substrate module” refers to biomaterial substrate modules that include a plurality of lattice sub-structures having identical (or substantially identical) geometries. Examples of homogeneous biomaterial substrate modules are shown as 100b, 100c and 100d in FIG. 3B. As used herein, the term “substantially” is used as a term of approximation, and not as a term of degree, and is intended to account for inherent variations in measurements and measurement methodologies. For example, “substantially identical” in this context refers to the geometries being identical within an acceptable level of measurement error such that, for example, the cell phenotype resulting from the two lattice sub-structures is not meaningfully different.

The lattice sub-structures 110 contributing to the functionally graded biomaterial substrate module 100 may be fabricated by any suitable method and using any suitable materials, without limitation. In some embodiments, for example, the lattice sub-structures 110 may be fabricated using the methods, materials and dimensions/parameters described in U.S. patent application Ser. No. 15/998,685 (now U.S. Pat. No. 11,078,459 issued Aug. 3, 2021), titled “Integrated methods for precision manufacturing of tissue engineering scaffolds,” to Tourlomousis et al. (assigned to the same Assignee as the present application), the entire content of which is incorporated herein by reference. The individual lattice sub-structures 110 can also be fabricated with any suitable geometrical parameters, as also disclosed in U.S. patent application Ser. No. 15/998,685 (now U.S. Pat. No. 11,078,459 issued Aug. 3, 2021), titled “Integrated methods for precision manufacturing of tissue engineering scaffolds,” to Tourlomousis et al. (assigned to the same Assignee as the present application), the entire content of which is incorporated herein by reference. For example, as disclosed in U.S. patent application Ser. No. 15/998,685 (now U.S. Pat. No. 11,078,459 issued Aug. 3, 2021), titled “Integrated methods for precision manufacturing of tissue engineering scaffolds,” to Tourlomousis et al. (assigned to the same Assignee as the present application), the entire content of which is incorporated herein by reference, the filaments may be constructed of a polymer, polymeric gel or suspension, such as, but not limited to, polycaprolactone (which is already approved by Federal Drug Administration for in vivo applications).

In some embodiments, the lattice sub-structures can be fabricated according to the melt electrowriting method described in U.S. patent application Ser. No. 15/998,685 (now U.S. Pat. No. 11,078,459 issued Aug. 3, 2021), titled “Integrated methods for precision manufacturing of tissue engineering scaffolds,” to Tourlomousis et al. (assigned to the same Assignee as the present application), the entire content of which is incorporated herein by reference, from filaments that are as small as 10 micrometers (as well as larger filaments, for example, but not limited to, filaments ranging from 10 to 100 micrometers). The electrowriting method prints lattice sub-structures having porous, fine mesh-like structures with geometric features that are generally at the same size scale as the cells themselves, enabling the cells to form adhesions to the lattice structures. This allows the resulting cell shapes to be controlled by adjusting the microarchitecture (i.e., geometry) of the printed lattice sub-structure, as described herein. The melt electrowriting (MEW) technique is described in detail in U.S. patent application Ser. No. 15/998,685 (now U.S. Pat. No. 11,078,459 issued Aug. 3, 2021), titled “Integrated methods for precision manufacturing of tissue engineering scaffolds,” to Tourlomousis et al. (assigned to the same Assignee as the present application), the entire content of which is incorporated herein by reference. This method is an extremely fine-scale form of 3-D printing which uses an electric field “electrospinning” to draw fibers that have diameters of about 10 to about 100 microns while precisely controlling the geometry (i.e., the lattice geometry) of the lattice sub-structure via 3-D printing. By comparison, conventional 3-D printing can produce porous lattice geometries with precisely controlled geometries only with filaments no smaller than 150 micrometers.

As discussed in U.S. patent application Ser. No. 15/998,685 (now U.S. Pat. No. 11,078,459 issued Aug. 3, 2021), titled “Integrated methods for precision manufacturing of tissue engineering scaffolds,” to Tourlomousis et al. (assigned to the same Assignee as the present application), the entire content of which is incorporated herein by reference, the melt electrowriting (MEW) technique allows precise control of the porous microarchitecture of a 3-D scaffold with cellular-relevant geometrical feature sizes, providing control of the shapes and the phenotypes of the expanded stem cells. These methods enable generation of the desired types of scaffold geometry in a reproducible and industrially scalable manner. In some embodiments, the techniques combine melt electrospinning and additive manufacturing. Embodiments of this manufacturing method (designated hereinafter as “the TCK method”) may be used to fabricate scaffold meshes of unmatched geometrical fidelity and precision. And embodiments of the TCK method may be used to fabricate novel scaffold designs involving, for example, 0-90 and 0-45 degree (among others) fibrous architectures with consistent fiber diameters, orientations, alignment, and interconnectivities. The TCK method may utilize Melt Electrospinning Writing (MEW) to

manufacture the integrated scaffolds. In some embodiments, Poly(e-polycaprolactone) (PCL) is selected for MEW because it is approved by the US Food and Drug Administration for in vivo applications, and because it is biocompatibile, long-term biodegradable, and has a relatively low and wide melt processing temperature window (60°-90° C.). Any suitable polycaprolactone may be used without limitation. For example, in some embodiments, PCLs having material specifications with an average molecular weight of 45,600 g/mol and polydispersity of 1.219 can be used. An example PCL may be obtained, for example, from Perstorp Ltd. of Warrington, UK (Capa6500). However, it is also understood that any suitable biopolymer may be used, and the present disclosure is not limited to polycaprolactones.

In some embodiments, for example, PCL pellets may be molded into 8 mm and 25 mm circular disks using aluminum shims between Teflon surfaces and a Carver press at 120° C. for subsequent rheological characterization. In some embodiments, this can be accomplished with the advanced rheological extended system (ARES) of Rheometric Scientific (currently TA Instruments) in conjunction with stainless-steel parallel disk fixtures with 25 mm diameter for small-amplitude oscillatory shear (SAOS) and steady torsional flow experiments. The force-rebalance transducer of the rheometer is capable of measuring simultaneously both the normal force and the torque. The oven temperature of the rheometer is controlled within±0.1 ° C. The rheological characterization experiments can be carried out at 70° C., 80° C., and 90° C. and using a constant 1 mm gap.

In some embodiments, a high-resolution heat-assisted MEW system configuration can be established. The process design may be guided by detailed characterization of the thermo-rheological processing properties of the biomaterial substrate along with the fluid dynamics, heat transfer, and electrostatics multiphysics phenomena governing the process under investigation. The overall system configuration may be analyzed based on three defined discrete process regimes.

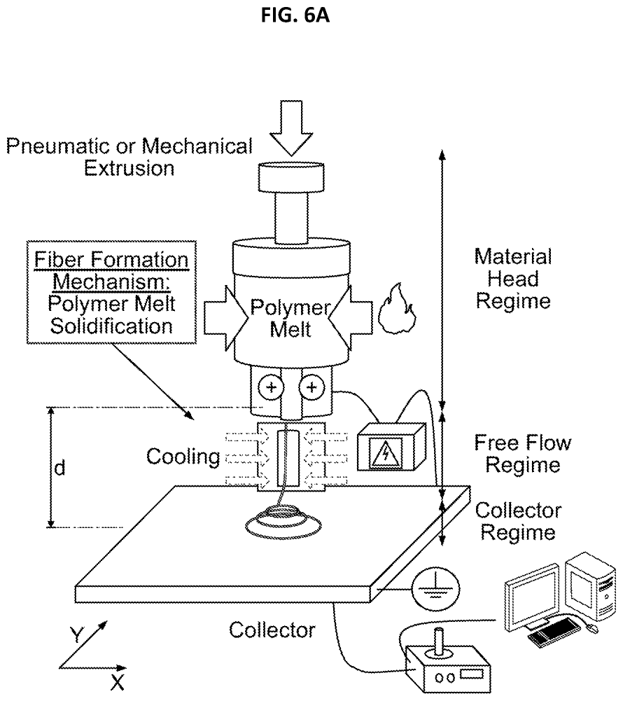

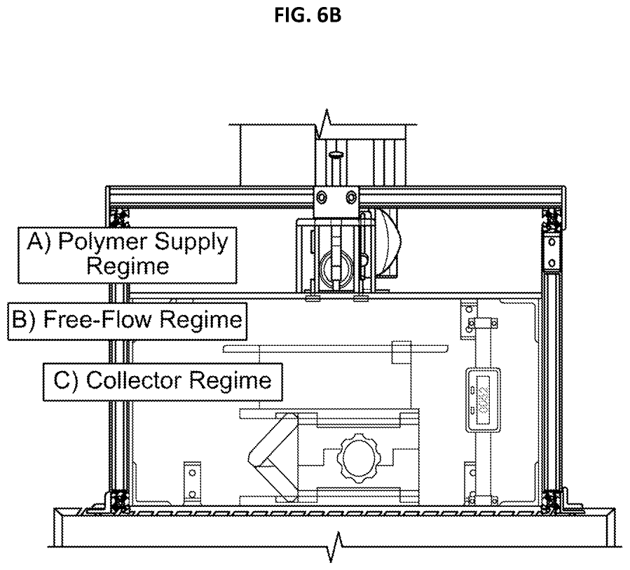

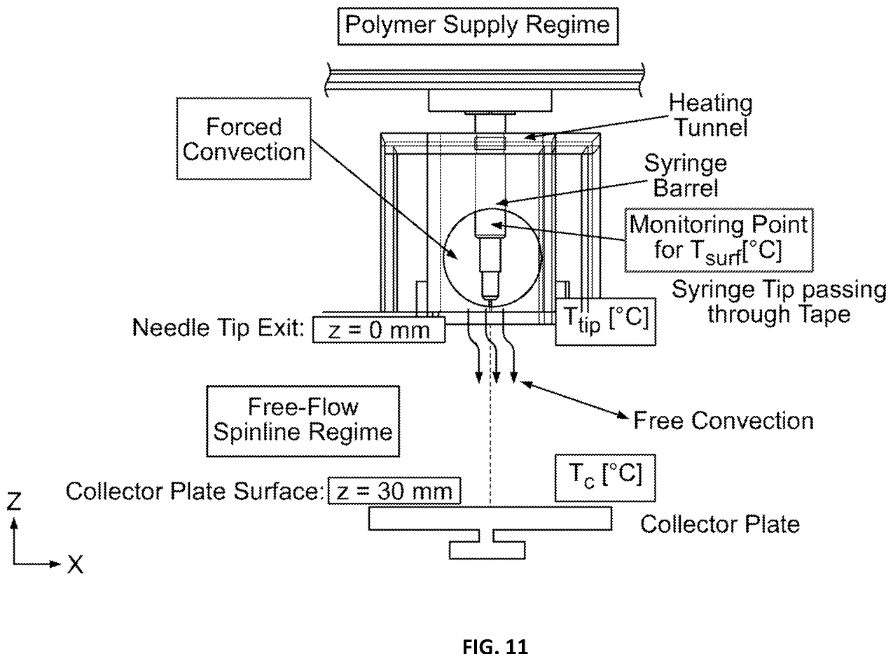

First, the polymer melt supply regime can be composed of a glass Luer-lock 5 ml syringe (such as that available for purchase from Hamilton, Reno, NV) and a stainless-steel needle tip with a plastic hub (such as that obtainable from McMaster Carr, Elmhurst, IL) attached to it. The polymer melt can be maintained in a uniform melt state using an industrial heat gun (e.g., Steinel, HG 2510 ESD). In addition, a programmable syringe pump (such as that obtained from Harvard Apparatus, Holliston, MA) may be mounted vertically and used to set the volumetric flow rate by adjusting the speed of the plunger within a syringe (flow accuracy being within 0.25% and reproducibility being within 0.05%). The temperature can be monitored both at the syringe barrel and the capillary tip with an infrared FUR thermal camera (such as the PM 290, Inframetrics, Thermacam). In the free-flow regime, a high-voltage source (a suitable source can be acquired from Gamma High Voltage Research, Ormond Beach, FL) may be used for the application of a voltage potential between the needle tip and a grounded electrically conductive collector. An aluminum collector may be mounted on an x-y programmable stage (such as that obtainable from ASI Applied Scientific Instrumentation, Eugene, OR) that is sequentially mounted on a lab jack (obtained, e.g., from Newport Corporation, Irvine, CA) (See FIGS. 6A, 6B and 7-11). The distance between the tip and the collector plate can be monitored using a vertical digital meter (FIGS. 6A, 6B and 7-11) and set manually using the lab jack's turning knob with a vertical positioning resolution of 0.5 mm. To compensate for ambient conditions that might affect the process, the overall system configuration may be placed on an anti-vibrating optical table with the spinning apparatus contained within a plexiglass enclosure. Furthermore, the temperature and humidity values within the enclosure can be monitored using a multimeter (such as that which can be acquired from Extech Instruments, Waltham, MA) equipped with a type K thermocouple.

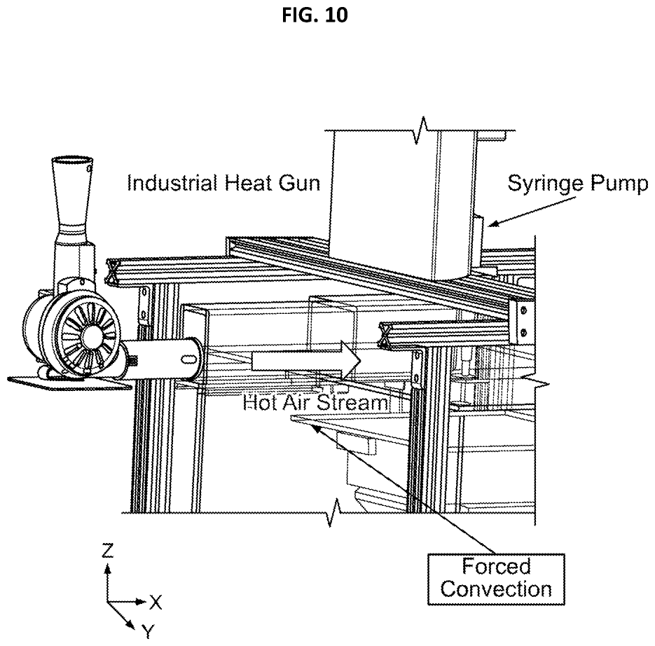

The heating element may be composed of an industrial heat gun (HG) with controllable air flow (QHG) (0.002-0.008 m³/s) and adjustable air temperature (THG) settings (49°-649° C.). The heat gun may be mounted at the entrance of a heating tunnel housed by a transparent chamber constructed out of poly (methyl methacrylate) (FIG. 10). The syringe may pass through the heating tunnel, and a small portion of the syringe needle tip may reach the interior of the chamber through an electrically conductive tape covering a circular opening created at the ceiling of the chamber. Heating insulation tapes may be applied onto the back wall and the floor of the heating tunnel in order to minimize heat losses. The area of the circular opening covered by the tape may be kept tightly sealed in order to avoid disturbances along the spin-line regime from the hot stream air.

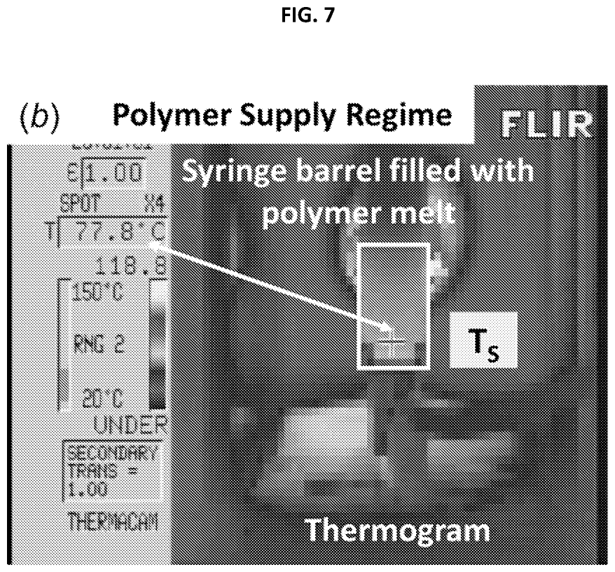

The surface of the syringe may be heated due to heat transfer via forced convection generated by the heat gun, and the ambient temperature conditions along the spin-line are governed by free convection through to the heated tape. The heat transfer conditions may be calibrated so that the temperature at the surface of the syringe hosting the PCL melt may be maintained as the desired temperature. For example, it may be determined that for the air flow rate of Q_{HG}=0.0017 m³/s and air temperature of T_{HG}=132° C., the temperature on the syringe surface (Ts) may be set and maintained at 78±1° C. (FIG. 7). Thermal imaging using the FUR camera can confirm that the temperature at the surface of the syringe does not vary outside of the Ts±1° C. over the time course.

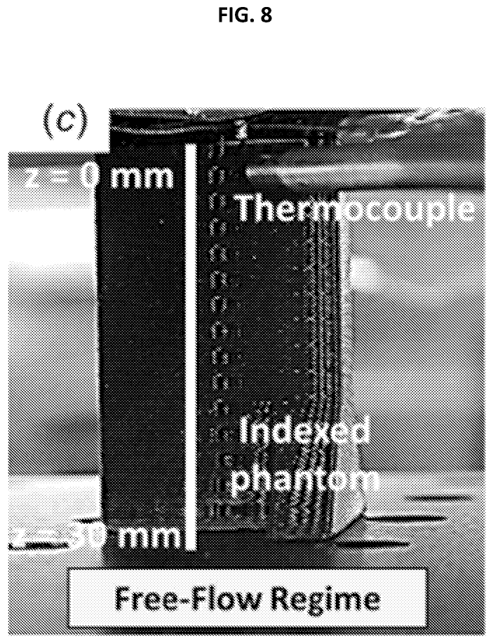

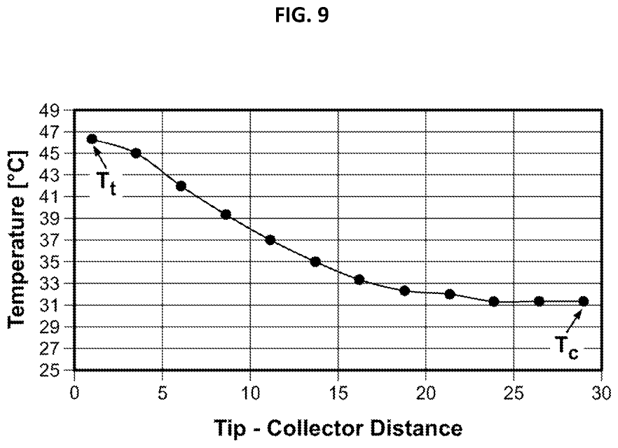

Thermocouple measurements along the spin-line coordinate z (FIG. 8), where z=0 mm may be considered a measurement point under the tip (Tt=40±5° C.) and z=30 mm may be considered a measurement point on the surface of the collector plate (Tc=30±5° C.), demonstrate the presence of an exponentially decaying temperature profile (FIG. 9). Due to the high thermal conductivity of glass and the small volume of polymer melt hosted in the syringe barrel, it may be assumed that the temperature of the polymer melt (Tₒ) becomes equal to Tₛ, and the system reaches thermal equilibrium after 1 h. The latter may also be confirmed by measuring a stable spin-line temperature profile regularly after the heat gun is set over the time course of 2 h. In this way, the presence of temperature gradients higher than 5° C. along the process regimes that may yield variations in the temperature-dependent polymer viscosity, and thus in the flow field along the process regimes, may be avoided.

As demonstrated in U.S. patent application Ser. No. 15/998,685 (now U.S. Pat. No. 11,078,459 issued Aug. 3, 2021), titled “Integrated methods for precision manufacturing of tissue engineering scaffolds,” to Tourlomousis et al. (assigned to the same Assignee as the present application), the entire content of which is incorporated herein by reference, a heat-gun based system is capable of maintaining uniform heating within the material head and a spin-line temperature profile, whose higher end can be set close to the onset crystallization temperature of the biopolymer (here, PCL). This capability can offer an alternative way of printing aligned fibers with submicron diameter by tuning the spin-line temperature so as to induce prolonged stretching, through delayed “in-flight” fiber solidification.

Prior to the printing, pure biopolymer (e.g., PCL) pellets are loaded into a glass syringe (e.g., Hamilton). Then, the syringe may be placed in a laboratory convective oven and heated for 24 h to remove any bubbles that may affect the process stability and downstream structural formability of the melt electrospun fibers. After assuring the homogeneity of the polymer melt, a needle tip at a prescribed nominal inner diameter (21 gauge-0.514 mm) may be adapted onto the syringe. The syringe with the attached tip may then be placed in the material head of the system, which may be preheated at a temperature (T_{surf}=77.8° C.) with the heating element. At least 1 h may be given to the system prior to initiating printing in order to reach thermal equilibrium.

According to embodiments of the present disclosure, the operating conditions to be used during the electrowriting process are not particularly limited, and can be selected on the basis of the dimensionless analysis of process parameters, and the resulting bioprintability numbers, as discussed in U.S. patent application Ser. No. 15/998,685 (now U.S. Pat. No. 11,078,459 issued Aug. 3, 2021), titled “Integrated methods for precision manufacturing of tissue engineering scaffolds,” to Tourlomousis et al. (assigned to the same Assignee as the present application), the entire content of which is incorporated herein by reference. This procedure is further described in Chang et al., “Melt electrospinning writing process guided by a “Printability Number””, ASME Journal of Manufacturing Science and Engineering, 139, 081004-1-15 (2017). doi:10.1115/1.4036348.

For example, as discussed in in U.S. patent application Ser. No. 15/998,685 (now U.S. Pat. No. 11,078,459 issued Aug. 3, 2021), titled “Integrated methods for precision manufacturing of tissue engineering scaffolds,” to Tourlomousis et al. (assigned to the same Assignee as the present application), the entire content of which is incorporated herein by reference, the identification of relevant dimensionless groups can be carried out using a classical dimensional analysis technique starting with process and system-specific independent parameters. The following definitions are employed. “n” is the number of independent variables relevant to the process. “j” is the number of base dimensions found in the n variables. “Y” is the number of variables necessary to be considered simultaneously. “k” is the number of the independent π terms that can be identified to describe the process and is equal to n−j (i.e., k=n−j).

The total number of independent variables, n, is equal to 12. Table 1, below, enumerates these variables and their base dimensions, where M stands for mass (SI unit: kilogram), L stands for length (SI unit: meter), T stands for time (SI unit: second), Θ stands for temperature (SI unit: Kelvin), and A stand for electric current (SI unit: Ampere).

TABLE 1
List of independent variables along with base dimensions: j = {L, M, T, A, Θ}

| | | | | | | | | | | | | |
|---|---|---|---|---|---|---|---|---|---|---|---|---|
| Variables | Rₒ | d | Q | Vₚ | Tₜ | Tₒ | ρ | η | ε | γ | λ | g |
| SI units | m | m | m³/s | V | K | K | kg/m³ | Pa · s | $\frac{F}{m}$ | $\frac{N}{m}$ | s | Kg/m³ |
| Equivalent | — | — | — | Kgm²s⁻³A⁻¹ | — | — | — | mkgs⁻¹ | s⁴A²kg⁻¹m⁻³ | kgs⁻² | — | — |
| with more | | | | | | | | | | | | |
| basic SI | | | | | | | | | | | | |
| units | | | | | | | | | | | | |
| Base | L | L | L³T⁻¹ | ML²T⁻³A⁻¹ | Θ | Θ | ML⁻³ | L⁻¹MT⁻¹ | T⁴A²M⁻¹L⁻³ | MT⁻² | T | LT⁻² |
| dimensions | | | | | | | | | | | | |

This number of base dimensions is equal to 5 with j′={L, M, T, A, Θ}. Next, j is determined by assuming that j=j′ and scanning for j repeating variables which do not form a dimensionless product. The prescribed number of five independent variables leads to the following independent variables)={d, Q, Vp, T_{I}, y}. Thus, the number of independent dimensionless π terms that could be formed would be equal to k=n−j=12−5=7. The following step consists of the πᵢ, i=1, 2 . . . 7 term formation. Each term is formed by forming a power product of the j repeating variables with the additional variable.

The procedure followed for P1 term formation is shown in Equation (1):

π₁=Rₒ^{α}^{₁}g^{α}^{₂}ϵ^{α}^{₃}Tₜ^{α}^{₄}γ^{α}^{₅}ηₚ⁻¹ (1)

Then, the dimensions of the various quantities are inserted inside Eq. (1) as shown in Equation (2):

dimension of π₁=L^{α}^{₁}⁺^{₂}^{-3α}^{₃}M^{-α}^{₃}^{+α}^{₅}⁻¹T^{-3α}^{₂}^{+4α}^{₃}^{-2α}^{₅}⁺¹A^{2α}^{₃}Θ^{α}^{₄} (2)

To obtain a dimensionless parameter π, each exponent M, L, T, etc., needed to vanish, thereby yielding a system of linear algebraic equations, Equations (3)-(7):

α₁+α₂−3α₃=0 (3)

−α₃+α₅=1 (4)

−2α₂+4α₃−2α₅=−1 (5)

2α₃=0 (6)

α₄=0 (7)

The solution of the system (Eqs. (3)-(7)) and its subsequent substitution in Eq. (1) yields a dimensionless term Tri shown in Table 2, below. The same procedure is followed for the formation of the remaining u terms shown in Table 2. Thus, the product combination of the πᵢ dimensionless terms can lead to a single dimensionless number π.

π=π=π₁*π₂* . . . *π₇ (8)

TABLE 2
List of dimensionless Π₁, = 1, 2 ... , 7 terms

| Π₁ | Π₂ | Π₃ | Π₄ | Π₅ | Π₆ | Π₇ |
|---|---|---|---|---|---|---|
| $\frac{{d}^{1 / 2} γ}{{g}^{1 / 2} {η}_{p}}$ | $\frac{{T}_{t}}{{T}_{C}}$ | $\frac{{R}_{o}}{d}$ | $\frac{{d}^{5 / 2} {g}^{1 / 2}}{Q}$ | $\frac{g {ε}^{1 / 2} {V}_{p}}{{d}^{3 / 2} {γ}^{3 / 2}}$ | $\frac{{d}^{1 / 2}}{{g}^{1 / 2} λ}$ | $\frac{γ}{{d}^{2} dp}$ |

Substituting for each individual π term from Table 2 yields the following dimensionless π number, denoted as N₁ herein:

$${N}_{1} = \frac{{y}^{1 / 2} {ε}^{1 / 2}}{{g}^{1 / 2}} \frac{{T}_{t}}{λρ} \frac{{R}_{0}}{Tc} \frac{{V}_{p}}{d {Qn}_{p}} \qquad ( 9 )$$

To account for the translational stage speed U_{T} as an independent parameter, an additional dimensionless group π₈ is formulated as an additional multiplier in Eq. (8):

$${\prod }_{8} = \frac{{U}_{T}}{{R}_{o}^{1 / 2} {g}^{1 / 2}} \qquad ( 10 )$$

yielding the following N₂ term:

$${N}_{2} = \frac{{γ}^{1 / 2} {ε}^{1 / 2} {R}_{o}^{1 / 2}}{g λρ} \frac{{T}_{t}}{{T}_{C}} \frac{{V}_{p} {U}_{T}}{{dQn}_{p}} \qquad ( 11 )$$

The formulation and calculation of two separate terms, N₁ and N₂, in turn enable the investigation of printability when the process is performed under a stationary (U_{T}=0) and a moving collector (U_{T}>0), respectively. In the former case, the N₁ term is a function of the independent process parameters that govern the polymer melt jet formation in the free-flow regime. In the latter case, the N₂ term additionally accounts for the translational stage speed (U_{T}), a process variable that quantitatively affects the fiber topography on the receiving substrate. To be sure, the initial N₁ term is defined for the preliminary procedural step of identifying the equilibrium state conditions in the free-flow regime to ensure stable jet formation. In the absence of this preliminary step, the direct application of N₂ for a stationary collector may yield a trivial printability value of zero.

What follows is a set of nondimensionalized equations that enable the identification of the important dimensionless groups that can be tuned toward efficient printability.

A thin filament approximation is used, and by focusing on a small part of the melt electrospun stable jet region, a one-dimensional momentum balance may be made by considering the various forces affecting the jet profile. The jet is subjected to: (a) Coulombic electrostatic, viscous, elastic, surface tension, and gravitational forces. Assuming axisymmetry along the path from the tip of the spinneret up to the surface of the collector (at distance, d) and using the characteristic quantities defined in Table 3, below, the dynamics of the melt electrospun jet can be modeled using the following system of nondimensional equations, where R is the jet radius divided by the characteristic jet radius Rₒ just outside of the needle tip, v is the jet velocity divided by the characteristic velocity vₒ, R is the jet radius, and the prime indicates derivatives with respect to the spin-line coordinate z:
A(1) Conservation of mass—Continuity:

R²v=1
A(2) Conservation of momentum:

$${Revv}^{′} = Bo + 3 ( 1 - {r}_{n} ) \frac{( {R}^{2} {v}^{′} )}{{R}^{2}} + \frac{{T}_{p}^{′}}{{R}^{2}} + Ca \frac{{R}^{′}}{{R}^{2}} + {E}_{p} ( {σσ}^{′} + β {EE}^{′} + \frac{2 E σ}{R} )$$

where Re, Bo, Ca, and Ep are defined in Table 3.
A(3) Conservation of charge:

σ=R
A(4) Electric field:

$${E}_{t} = \frac{1}{( 1 + 2 z - {z}^{2} / χ ) \sqrt{1 + {( {R}^{′} )}^{2}}}$$

The viscoelastic nature of the polymer melt is taken into consideration by the use of the Giesekus model, which expresses the viscous polymer stress τₚ in terms of the applied deformation, which is represented by the strain rate tensor γ:

$$A ( 5 ) {τ}_{p} + {λτ}_{p ( 1 )} - α \frac{λ}{{n}_{p}} \{ {τ}_{p} \cdot {τ}_{p} \} = - {n}_{p} \dot{γ}$$

The viscous polymer stress τₚ denotes the elastic nature of the material due to normal stresses that arise during its deformation, and the strain rate tensor γ is given by the sum of the velocity gradient and its reciprocal. The input parameters of the Giesekus model that are determined by fitting the experimental raw data on the basis of the corresponding rheological material functions for each type of tested viscometric flow are the following: ηₚ represents the polymer viscosity parameter, λ the relaxation time, and α the mobility factor, which is a parameter related to the anisotropic Brownian motion and/or hydrodynamic drag on the constituent polymer molecules.

The nondimensional components of the viscous polymer stress tensor τₚ are given based on the constitutive Giesekus model (Eq. (1)) in axisymmetric cylindrical coordinates as:

$$A ( 6 ) {τ}_{p , rr} + De ( v {τ}_{p , rr}^{′} + {v}^{′} {τ}_{p , rr} ) + α \frac{De}{{r}_{n}} {τ}_{p , rr}^{2} = - {r}_{n} {v}^{′} \\ A ( 7 ) {τ}_{p , zz} + De ( v {τ}_{p , zz}^{′} - 2 {v}^{′} {τ}_{p , zz} ) + α \frac{De}{{r}_{n}} {τ}_{p , zz}^{2} = 2 {r}_{n} {v}^{′}$$

These dimensionless numbers calculated using the above equations are further summarized in Table 3 below.

TABLE 3
Characteristic quantities along with nondimensional numbers obtained
based on the governing equations

| Characteristic quantities | | |
|---|---|---|
| Length | R₀ | |
| Velocity | ${v}_{o} = \frac{Q}{π {R}_{o}^{2}}$ | |
| Electric Field | ${E}_{o} = E ( 0 ) = \frac{2 {V}_{p}}{{R}_{o} ln ( 1 + 4 d / {R}_{o} )}$ | |
| Dimensionless groups and their definitions Bond number | $Bo = \frac{ρ {gR}_{o}^{2}}{{η}_{o} ( {T}_{m} ) {v}_{o}}$ | $( \frac{gravity}{inertia} )$ |
| Electrostatic force parameter | ${E}_{p} = \frac{{ε}_{o} {E}_{o}^{2} {R}_{o}}{{η}_{o} ( {T}_{m} ) {v}_{o}}$ | $( \frac{electrostatic}{inertia} )$ |
| Capillary number | $Ca = \frac{{η}_{o} ( {T}_{m} ) {v}_{o}}{γ}$ | $( \frac{inertia}{surface tension} )$ |
| Reynolds number | $Re = \frac{ρ {v}_{o} {R}_{o}}{{η}_{o} ( {T}_{m} )}$ | $( \frac{inertia}{viscous} )$ |
| Deborah number | $De = \frac{λ {v}_{o}}{{R}_{o}}$ | $( \frac{relaxation time}{time scale of flow} )$ |

Initiation of the printing process requires: (a) droplet emergence, (b)

successful Taylor cone formation, and (c) subsequent emergence of a charged jet, which is electrostatically drawn across the spin-line coordinate in the free-flow regime. All phenomena are dependent on the relative importance of the forces applied at the polymer melt jet.

Downstream pulling forces such as the gravitational and the electrostatic Coulombic forces are related to the Bond (Bo) number and the electrostatic force parameter (Ep), respectively. Upstream resistive forces such as the viscous, the elastic, and the surface tension forces are related to the Reynolds (Re) number, the Deborah (De) number, and the Capillary (Ca) number. According to the electrospinning operating principle, Taylor cone formation occurs when the electrostatic forces overcome the capillary forces. Jet initiation and the electrostatic drawing of the polymer melt jet are strongly dependent on the viscoelasticity of the polymer melt. If the gravitational forces, along with the electrostatic drawing forces caused by the accumulation of the charges at the jet-ambient air interface, overcome the viscous and elastic stresses that are applied to the polymer melt, jet initiation occurs. Thus, the proposed Printability Number should assume values within a domain defined by a set of independent material, process, and geometry-related parameters for which the printing process can be realized.

A dimensional analysis may be employed based on measurable polymer properties and controllable process parameters. Consistent with standard engineering practice, simplified dimensionless numbers may be derived by taking the product of the formulated ones. For example, seven dimensionless groups may be formulated (π_{1, 2 . . . 7}) based on the procedure detailed above. To this end, the N₁ number given by Eq. (9) may be defined as the “Printability Number” for a stationary collector and denoted as N_{PR,1}:

$${N}_{PR , 1} = \frac{{γ}^{1 / 2} {ε}^{1 / 2}}{{g}^{1 / 2} λρ} \frac{{T}_{t}}{{T}_{C}} \frac{{R}_{o}}{d} \frac{{V}_{p}}{Q {η}_{p} ( {T}_{m} )} \qquad ( 12 )$$

where ηₚ(Tₘ) denotes the melting temperature dependency of the polymer viscosity, and the characteristic jet radius just outside the needle tip, R₀, is assumed to be equal to the needle tip diameter.

Material functions of the Giesekus model may be used for nonlinear fitting of experimental data and determination of model-specific input parameters for the polymer melt to be processed. The values of the loss modulus, G″, i.e., the energy dissipated as heat, have been shown to be higher than the values of the storage modulus, G′, i.e., the energy stored as elastic energy, over a broad range of frequencies for a certain PCL during MEW processing. In the linear viscoelastic region, i.e., relatively small strains and strain rates as would be encountered at the relatively low flow rate conditions of the melt electrospinning writing process (<50 μL/h), the shear viscosity of the polymer melt can be considered to be Newtonian (i.e., the zero-shear viscosity, η₀(Tₘ)). Up to a shear rate of 10 s⁻¹ the shear viscosity of PCL is constant. In the linear viscoelastic region, the uniaxial extensional viscosity of the melt, i.e., the Trouton viscosity, is equal to three times the Newtonian (zero-shear) viscosity, η₀(Tₘ)

ηₚ(Tₘ)=3ₒ(Tₘ) (13)

Substituting the Trouton viscosity into Eq. (12) yields the following Printability Number, N_{PR,1}:

$${N}_{PR , 1} = \frac{1}{3} \frac{{γ}^{1 / 2} {ε}^{1 / 2}}{{g}^{1 / 2} λρ} \frac{{T}_{t}}{{T}_{C}} \frac{{R}_{o}}{d} \frac{{V}_{p}}{Q {η}_{p} ( {T}_{m} )} \qquad ( 14 )$$

The zero-shear viscosities obtained from the rheological data for three different melting temperatures (Tm=70, 80, and 90° C.) are fitted using an Arrhenius type equation in order to obtain the activation energy of flow (ΔH/R_{ig}) (SI:K)

$${η}_{o} ( {T}_{m} ) = {η}_{o} ( {T}_{ref} ) exp [ \frac{Δ H}{{R}_{ig}} ( \frac{1}{{T}_{m}} - \frac{1}{{T}_{ref}} ) ] \qquad ( 15 )$$

where ΔH is the activation energy (SI: J/mol), R_{ig} is the universal gas constant (SI: J/K mol), and T_{ref} is the reference temperature. Substituting Eq. (15) into Eq. (14) yields the following definition of the Printability Number, N_{PR,1}:

$${N}_{PR , 1} = \frac{1}{3} \frac{{γ}^{1 / 2} {ε}^{1 / 2}}{{g}^{1 / 2} λρ} \frac{{T}_{t}}{{T}_{C}} \frac{{R}_{o}}{d} \frac{{V}_{p}}{Q {η}_{o} ( {T}_{ref} ) exp [ \frac{Δ H}{{R}_{ig}} ( \frac{1}{{T}_{m}} - \frac{1}{{T}_{ref}} ) ]} \qquad ( 16 )$$

N_{PR,1} can be computed using Eq. (16) for the melting range of PCL (70° C.≤Tm≤90° C.) and a prescribed set of typical process and material parameters. The values of the material parameters (summarized in Table 4, below) are either derived from literature or through fitting of the rheological data of the PCL used in processing for scaffold fabrication. In order to assure that N_{PR} assumes values within a valid domain, each range is determined based on previously reported studies where PCL has been successfully processed by way of MEW, and validated with the presently disclosed MEW system. Thus, a range of volumetric flow rates (25 μL/h≤Q≤50 μL/h) may be applied for a 21 gauge needle tip diameter (Dt=2·R₀), for collector distances (d) of 10 mm to 30 mm and a range of applied voltage potentials (10 kV≤Vₚ≤15 kV).

TABLE 4
Material properties of PCL used

| Parameters | Values | |
|---|---|---|
| Zero shear rate viscosity (at 78° C.) (ηₒ) | 3203 | Pa · s |
| Relaxation time (λ) | 0.08 | s |
| Activation energy of flow (ΔH/R_{ig}) | 4407.8 | K |
| Density of PCL (at 25° C.) | 1145 | kg/m³ |
| Surface tension coefficient (γ) | 30 | mN/m |

| | |
|---|---|
| Relative permittivity (εᵣ = ε/εₒ) | 3.1 |

The normalized N_{PR,1} is obtained by dividing the computed N_{PR,1} value with the N_{PR,1} value that defines the lower end of the printability window bounded by the material's melting range for T_{ref}=70° C. and Qₘₐₓ=50 μL/h. The temperature of the polymer melt inside the reservoir (T₀) is normalized with respect to the reference temperature (T_{ref}=70° C.), i.e., T*=Tₒ/T_{ref}. T*=Tₘ/T_{ref} since T₀ assumes the melt temperature value (Tₘ). The printability window is seen to depend significantly on the volumetric flow rate, with the smaller Q (25 μL/h) yielding significantly larger N_{PR,1} values compared to that obtained at the larger Q (50 μL/h). This trend is consistent with recent phenomenological observations that reflect stable printing by way of MEW under low volumetric flow rates. As T* increases within each printability window, N_{PR,1} increases exponentially due to the Arrhenius temperature dependence of the polymer melt viscosity, implying that for higher melt temperature conditions, the material can be electrospun more efficiently. This relationship indicates that for prescribed Dt, Q, and Vp settings, melt temperature conditions approaching the higher end of the material's melting temperature range (90° C. for PCL) enable earlier droplet emergence compared to the melt temperature conditions that approach the lower end of the material's melting temperature range, due to an increased volumetric flow rate inside the needle tip.

The N_{PR,1} formulation (Eq. (16)) implies that the electrical field strength (Vₚ/d) and the volumetric flow rate (Q) are the key independent parameters toward efficient printability (fiber mesh printing with consistent dimensional characteristics) provided that the melting and ambient conditions in the polymer melt supply regime and the temperature profile along the spin-line in the free-flow regime are not significantly perturbed during each printing event. N_{PR,1} scales as N_{PR,1}˜1/Q and N_{PR,1}˜VₚId. This validates the physical significance of the derived number that expresses the key combinatorial role of electrostatic, viscous, and inertial forces toward steady electrospinning conditions as previously demonstrated for solution-based electrospinning systems. Furthermore, all of the dimensionless groups are a function of Q-dependent inertial terms (see Table 3, above). Thus, the functional relationship between N_{PR,1} and each dimensionless number may be computed for the prescribed Q range and three different Vₚ values spanning the Vₚ range. The results may be plotted for N*_{PR,1} as a function of the Re, Ca, De, and Ep numbers revealing that upon prescribing the melting conditions, a unique printability window can be defined for each Vₚ setting.

In addition to the printability number, additional process parameters may be optimized or tailored to achieve acceptable or optimized printing. For example, as discussed in U.S. patent application Ser. No. 15/998,685 (now U.S. Pat. No. 11,078,459 issued Aug. 3, 2021), titled “Integrated methods for precision manufacturing of tissue engineering scaffolds,” to Tourlomousis et al. (assigned to the same Assignee as the present application), the entire content of which is incorporated herein by reference, Vₚ and Q may also be tailored or optimized. Such tailoring or optimization of these process parameters may aim to eliminate the perturbations observed under nonequilibrium processing conditions. Upon tailoring or optimization, for example, an equilibrium state, i.e., a state at which downstream pulling and upstream resistive forces during printing are balanced, may be achieved. To tailor these parameters, as a first step, the collector may be moved

closer to the needle tip (e.g., d=15 mm) to increase the electrical field strength. When the tip to collector distance d≤110 mm, arching may occur due to excess ionized air molecules and dry ambient conditions (humidity <25%). At such relatively small distances (d), the arching phenomenon may become more pronounced for applied voltages that are ≥15 kV. By reducing the distance (d), a higher electrical field intensity facilitates stretching of the excess material collected at the tip. However, solely reducing the distance (d) may not be sufficient to eliminate the periodic perturbations. To further eliminate the perturbations and achieve equilibrium conditions, a reduction in the volumetric flow rate (Q) may also be required. This may be suggested by the relative importance of the Q-dependent inertial forces with respect to the Vₚ-dependent electrostatic forces, as guided by the N_{PR,1} formulation denoted in Eq. (16). The decrease of the volumetric flow rate to Q=25 μL/h may result in the formation of a Taylor cone directly below the needle tip. However, chaotic jet movement may occur close to the collector plate, and stable jet may not be achieved. In order to eliminate the instabilities and establish equilibrium state conditions, the applied voltage potential can be decreased to 11.5 kV, yielding stable cone-jet formation for a period of 30 min after which the printing process could start, and lattice sub-structures may be printed.

In some embodiments, however, in addition to tailoring or optimizing the above described parameters, the translational stage speed may also be tuned or optimized to determine the critical stage speed (U_{CR}), at which aligned fibers can be deposited on a translating collector. At lower speeds (e.g., 2-8 mm/s), random fiber deposition may yield nonwoven structures typified by overlapping fibers with multiple fusion points. At intermediate translation speeds (e.g., 8-83 mm/s), repeatable coiling structures, for which the frequency of the overlap monotonically decreases as the stage speed increases, may be realized. When the translational stage speed reaches 83 mm/s, a well-aligned fiber with average diameter, D_{f}=23±1.5 μm (micrometer) could be printed on the collector. It should be noted that the changes in the translational stage speed may affect the drawdown of the fiber, and thus the changes in the resulting pulling force may have the potential to disturb the equilibrium condition, especially at UT»UCR. Thus, optimization of the translational stage speed may be carried out in conjunction with the tuning or optimization of N_{PR,1}. To this end, U_{T} may be incorporated as an additional independent parameter in the dimensional analysis, and the derived N₂ term (Eq. (11)) may be used as the Printability Number when the collector is moving. The modified Printability Number is denoted as N_{PR,2}, and its final form may be obtained by multiplying the N_{PR,1} with the π₈ term.

$${N}_{PR , 2} = \frac{1}{3} \frac{{γ}^{1 / 2} {ε}^{1 / 2} {R}_{o}^{1 / 2}}{g λρ d} \frac{{T}_{t}}{{T}_{C}} \frac{{V}_{p} {U}_{T}}{{Q}_{o} ( {T}_{ref} ) exp [ \frac{Δ H}{{R}_{ig}} ( \frac{1}{{T}_{m}} - \frac{1}{{T}_{ref}} ) ]} \qquad ( 17 )$$

A normalized Printability Number, N*_{PR,2}, may be obtained by dividing the computed N_{PR,2} value (based on Eq. (17)) with the N_{PR,1} value (based on Eq. (16)) that defines the lower end of the printability window bounded by the material's melting range for T_{ref}=70° C. and Qₘₐₓ=50 μL/h. The normalized Printability Number N*_{PR,2} may be computed for each fiber pattern where optimum printability is achieved when U_{T} is tuned to its critical value (U_{T}=U_{CR}).

With this setup and these calculations, interwoven fiber meshes can be

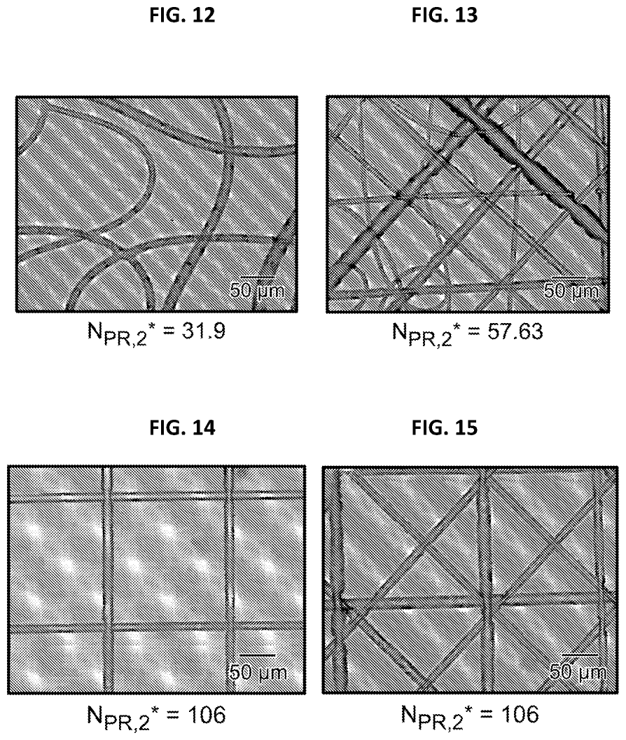

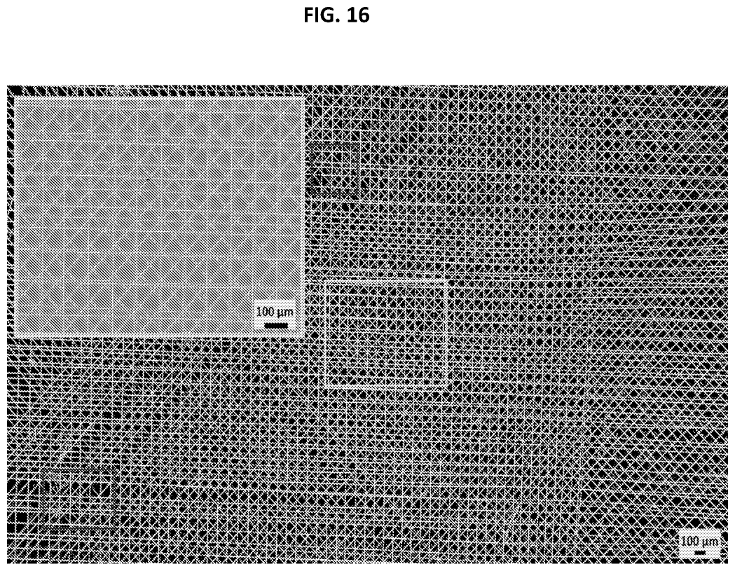

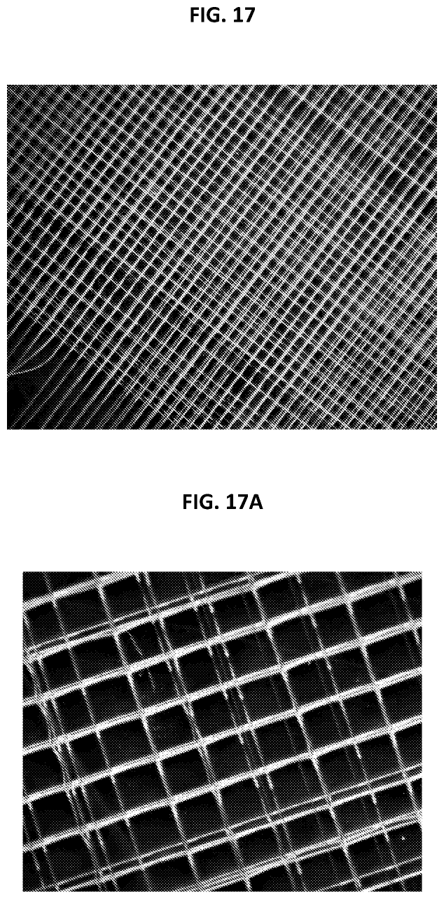

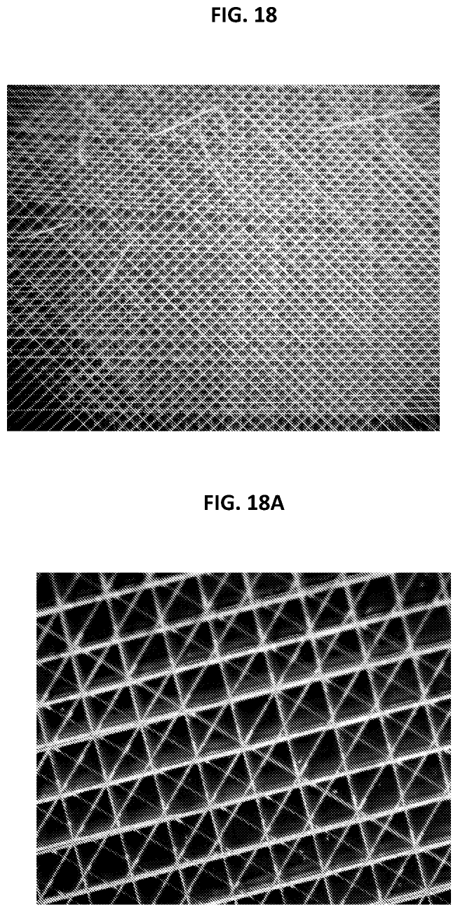

made for use as biological scaffolds (e.g., lattice sub-structures). Layered meshes with woven and nonwoven architectures may be fabricated using various N*_{PR,2} settings. Woven meshes with “0-90 deg” and “0-45-135-90 deg” pore architectures may be fabricated using optimized and non-optimized N*_{PR,2} settings. When N*_{PR,2} is not optimized, irregular structures may be observed. This is shown, e.g., in FIG. 12, which is obtained at an N*_{PR,2}=31.9, where U_{T}=25 mm/s<U_{CR}. When N*_{PR,2} is increased to 57.63 by independently tuning the stage speed (U_{T}=85 mm/s≥UCR) while neglecting equilibrium conditions in the freeflow regime, aligned structures with variable average fiber diameters (Df=27±14 μm) may be observed, as shown in FIG. 13. On the other hand, precise printing of mesh architectures composed of well-aligned fibers with uniform average diameters (D_{f}=23 μm±3.7 μm) can be produced for an optimal Printability Number of N*_{PR,2}=106. The produced fibers at this optimal printability setting are shown in FIGS. 14-18 bearing the hallmarks of equilibrium state conditions in tandem with appropriate tuning of U_{T} at its critical value.

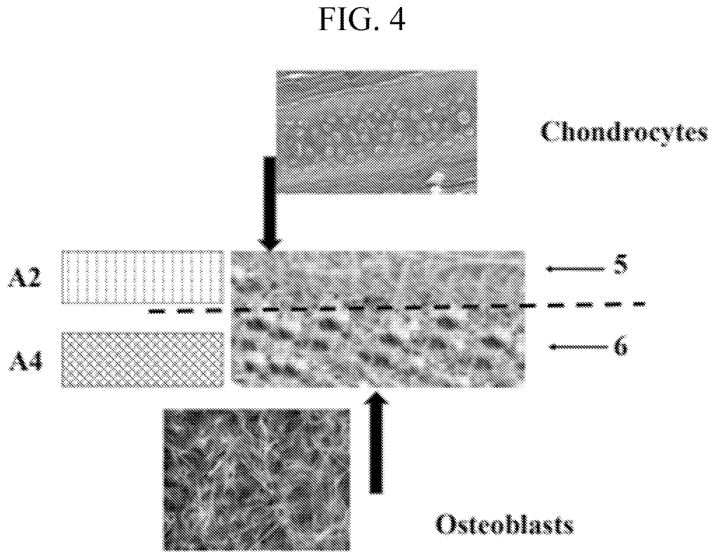

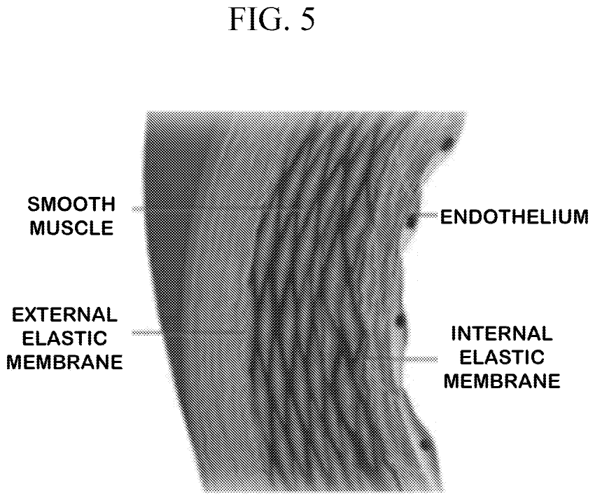

According to embodiments of the present disclosure, the lattice sub-structures may be assembled into a biomaterial substrate module 100 based on data from a programmable multimodal tissue fabrication database (as described further below) to fabricate a biomaterial substrate module 100 designed to yield a desired cell shape and/or long term tissue formation. For example, a plurality of different lattice sub-structures 110 having at least one different geometrical parameter may be assembled according to information from the programmable multimodal tissue fabrication database in order to create a biomaterial substrate module 100 designed to yield a particular cell shape, combination of cell shapes, tissue type or combination of tissue types. Similarly, a plurality of different biomaterial substrate modules 100 may be assembled according to information from the programmable multimodal tissue fabrication database in order to create a multi-module biomaterial substrate 200 designed to yield a particular combination of tissue types. These biomaterial substrate modules 100 and multi-module biomaterial substrates 200 can be used to direct formation of complex tissue formations (including, e.g., organ structures), e.g., an “interface tissue” as shown in FIG. 4 (with one layer yielding soft tissue, e.g., cartilage, and a second layer yielding harder tissue, e.g., cancellous or cortical bone), or multi-layered tissue formations as shown, e.g., in FIG. 5 (for example, a layer of endothelial cells yielding the formation of an endothelium, i.e., the formation of the linings of blood vessels when lumen-like structures are generated), and additional layers for muscle and elastic membranes generated by tissues obtained from differentiation of the stem cells into other phenotypes).

The lattice sub-structures 110 may be assembled or connected by any

suitable means or methods, without limitation, to form the biomaterial substrate modules 100. Similarly, the biomaterial substrate modules 100 may be assembled or connected by any suitable means or methods, without limitation, to form the multi-module biomaterial substrates 200. In some embodiments, however, the lattice sub-structures 110 may be fabricated (e.g., printed) according to the methods described above (and in U.S. patent application Ser. No. 15/998,685, titled “Integrated methods for precision manufacturing of tissue engineering scaffolds,” to Tourlomousis et al. (assigned to the same Assignee as the present application), the entire content of which is incorporated herein by reference). These lattice sub-structures 110 may then be assembled or connected in any manner to form the biomaterial substrate modules 100, and the biomaterial substrate modules 100 may then be assembled or connected to form the multi-module biomaterial substrates 200. Alternatively, the programmable multimodal tissue fabrication database may be used to generate a template (or instructions) for directly fabricating or printing the biomaterial substrate module 100 or multi-module biomaterial substrate 200, thereby omitting the need to “assemble” or “connect” multiple lattice sub-structures 110 to form biomaterial substrate modules 100, or to “assemble” or “connect” multiple biomaterial substrate modules 100 to form multi-module biomaterial substrates 200. In this way, the entire biomaterial structure (i.e., having multiple lattice sub-structures or multiple biomaterial substrate modules) can be achieved seamlessly by systematically altering the parameters of the printing (or manufacturing) process to fabricate the biomaterial structure in a contiguous, seamless manner.

The lattice sub-structures 110, biomaterial substrate modules 100 and multi-module biomaterial substrates 200 can have any three-dimensional shape, without limitation. Indeed, although shown in FIGS. 2B-D and 3B as generally rectangular or cubic in shape, the present disclosure is not limited to such shapes, and any shape may be used, e.g., other macroscopic shapes, including, but not limited to cylindrical, triangular, spherical, curvilinear and abstract shapes.

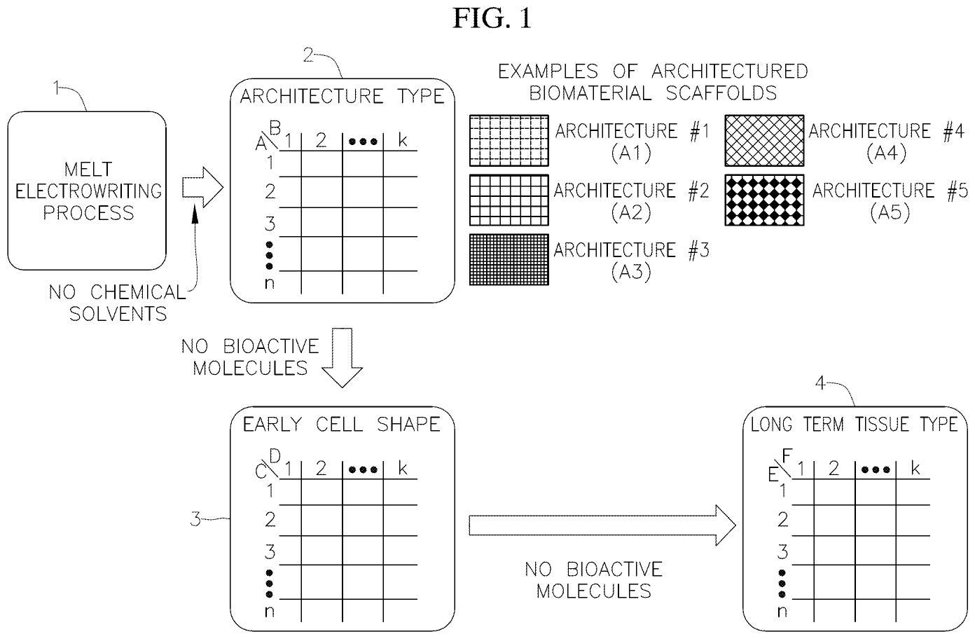

As discussed generally above, a programmable multimodal tissue fabrication database may be used to create a template for the biomaterial substrate modules 100 or the multi-module biomaterial substrates 200. In some embodiments, for example, as shown generally in FIG. 1, a method of determining the structure of a biomaterial substrate module 100 or multi-module biomaterial substrate 200 needed to create a particular tissue structure or formation includes generating (or accessing) a lattice sub-structure database, and generating a biomaterial structure map (e.g., a digital model or map) from the information in the lattice sub-structure database and information regarding the tissue or tissue formation intended to be fabricated using the biomaterial structure. The structure map (or digital model) of the biomaterial structure needed to form the desired tissue formation (or organ) can be constructed by mapping each type of tissue needed for different areas of the tissue formation, and digitally assembling the corresponding lattice sub-structure (or other biomaterial structure) identified by the database for each tissue type into the appropriate tissue formation in order to mimic the desired tissue formation. As the database houses biomaterial structure geometric data correlated to specific cell phenotypes or long term tissue type, the database can be used in this way to digitally construct a map (or digital model) that can then be used to physically construct the biomaterial structure needed to grow the mimicking tissue formation. As disclosed elsewhere herein, this digital map (or model) can be used as a printing map (or instructions) for direct printing by, e.g., a 3D printer, or as instructions for manual assembly or construction of the component lattice sub-structures or biomaterial substrate modules needed to complete the biomaterial structure defined by the map (or model). As used herein, the term “biomaterial structure” refers to the structures described herein to grow cells and/or tissue (e.g., tissue formations, including, e.g., organ structures), and may refer to the lattice sub-structures, the biomaterial substrate modules 100 and the multi-module biomaterial substrates 200 disclosed herein. As the structure needed to create the desired cell or tissue may differ, the term “biomaterial structure” is intended to denote that the needed structure dictated by the information from the database and the cell or tissue information may be any of the lattice sub-structures, biomaterial substrate modules, or multi-module biomaterial substrates, or combinations of these, disclosed herein.

The lattice sub-structure database may be generated by high-throughput screening and exploration of the wide variety of potential lattice geometries for the lattice sub-structures (which are described in U.S. patent application Ser. No. 15/998,685, titled “Integrated methods for precision manufacturing of tissue engineering scaffolds,” to Tourlomousis et al. (assigned to the same Assignee as the present application), the entire content of which is incorporated herein by reference. According to embodiments of the present disclosure, the database may be generated through parametric design and simulation of numerous lattice sub-structures having any number of lattice geometries. Within the database, the lattice sub-structures may be characterized by their fiber diameters, inter-fiber spacings and inter-layer angles. The ability to tightly control these 3 geometrical parameters allows the design and fabrication of lattice sub-structures spanning a large range of local and mechanical properties.

To enable the database to predict cell shape or tissue formation, the database correlates the lattice sub-structure geometry with the morphometric features of representative single cell shapes for each lattice sub-structure. For example, in some embodiments, generation of the database also includes fabrication of a representative set (or number) of lattice sub-structures (having a variety of different geometries), and seeding the fabricated lattice sub-structures with stem cells (e.g., mesenchymal stem cells from various sources and/or induced pluripotent stem cells). In some embodiments, the lattice sub-structures are fabricated without the use of chemical solvents, and the stem cells are seeded without the addition of bioactive molecules. The morphometric features of representative single cell shapes for each lattice sub-structure within 24 hours are then recorded in the database, thereby correlating (or mapping) lattice sub-structure geometry with the corresponding morphometric features of the single cell shapes. Metrology tools can be applied to determine the cell shapes within 24 hours of culturing, and positive and negative surface markers can be used to track cell phenotypes as a function of culturing time.

The database correlating lattice sub-structure geometries and corresponding cell phenotypes operates as a library identifying (or predicting) what lattice sub-structure shapes (or geometries) will generate what cell phenotypes. The database can also store information regarding the window of stability of the cell phenotypes.

To further enhance the database, the seeded lattice sub-structures are further monitored after the first 24 hours, and gene expression and differentiation of long-term tissue formation for each of the lattice sub-structures are recorded and correlated in the database. In some embodiments, again, no bioactive materials or molecules are used. As used herein, the term “bioactive” (as in “bioactive materials or molecules”) refers to a material or molecule that elicits a specific biological response at the interface of the bioactive material or molecule and the tissue which results in the formation of a bond between the tissue and the bioactive material or molecule. In some embodiments, only data corresponding to lattice sub-structures that yield a generally uniform tissue formation response are added to the database. In this way, the database enables mapping of lattice sub-structures with both single cell shape and long-term tissue formation.

Generation of the database is described in detail above, and in U.S. patent application Ser. No. 15/998,685, titled “Integrated methods for precision manufacturing of tissue engineering scaffolds,” to Tourlomousis et al. (assigned to the same Assignee as the present application), the entire content of which is incorporated herein by reference, and is therefore not repeated here. However, as a general discussion, confocal fluorescent microscopy is used to observe the single cell measurement outcomes of cells cultured on pre-fabricated lattice sub-structures. Examples of features that are probed include size, locations, and distributions of the attachment sites, or “focal adhesions,” i.e., subcellular protein-based complexes that various cells (including mesenchymal stem cells) make when cultured on substrates with various porous geometries. The large number of resulting images of cells under culture are analyzed and classified using artificial intelligence methods to correlate the cellular and subcellular features and their variability with various kinds of geometrical microenvironments that exhibit different spacings and arrangements of fibers. Quantifying the measurable features of the focal adhesions, a machine learning algorithm enables the classification of the shapes that cells assume during culture. Specifically, the advanced manufacturing approach was biologically qualified with a metrology framework that models and classifies cell confinement states under various substrate dimensionalities and architectures. See, e.g., paragraphs 0088 et seq. of U.S. patent application Ser. No. 15/998,685, titled “Integrated methods for precision manufacturing of tissue engineering scaffolds,” to Tourlomousis et al. (assigned to the same Assignee as the present application), the entire content of which is incorporated herein by reference.

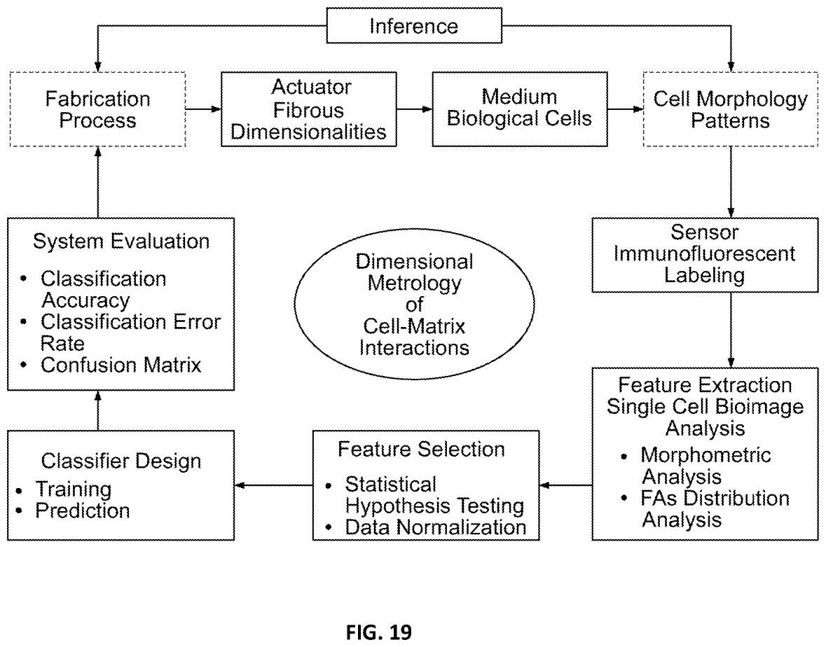

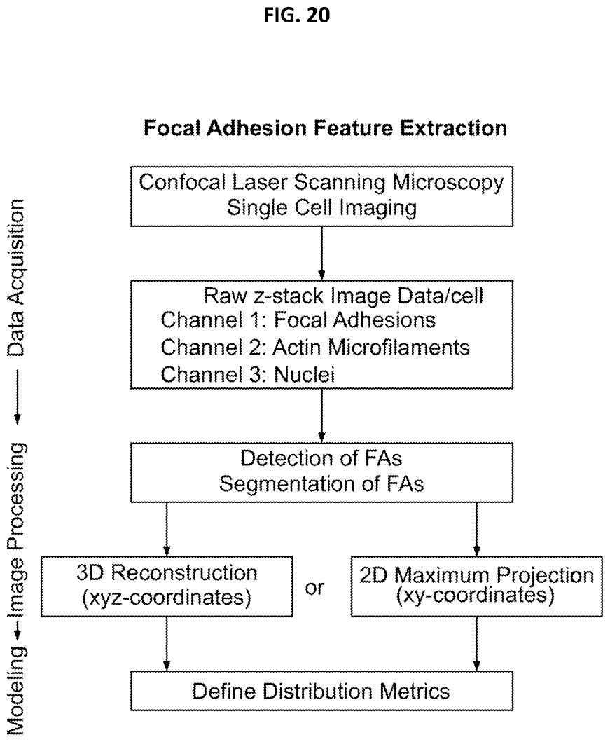

Indeed, as discussed more specifically in U.S. patent application Ser. No. 15/998,685, titled “Integrated methods for precision manufacturing of tissue engineering scaffolds,” to Tourlomousis et al. (assigned to the same Assignee as the present application), the entire content of which is incorporated herein by reference, the database may be generated using a method for quantitatively and reliably characterizing the measurements of cell position vectors and cell shapes. A block diagram of the metrology method is depicted in FIG. 19. Embodiments of the characterization method enable the rapid and reliable analysis and characterization of many cells under conditions of high throughput. Embodiments of the characterization method include immunofluorescent labeling of the cells for identification of structural and functional features with subsequent 3-D image acquisition, where the functional features include cell surface markers. Embodiments of the characterization method further include image analysis and automated algorithms for analyzing immunofluorescent-labeled cell features (FIG. 20), and generating statistics for the cell position and shape distributions that are then correlated with the cell phenotype (e.g., stem cell phenotype).

Embodiments of this method are referred to hereinafter as the “SIT” classification method. In embodiments of the present disclosure, the SIT classification method is integrated into an overall methodology of discovering the appropriate geometries for generating desirable cell shapes and phenotypes during the expansion of seeded cells.

According to embodiments, images of a cell may be generated via

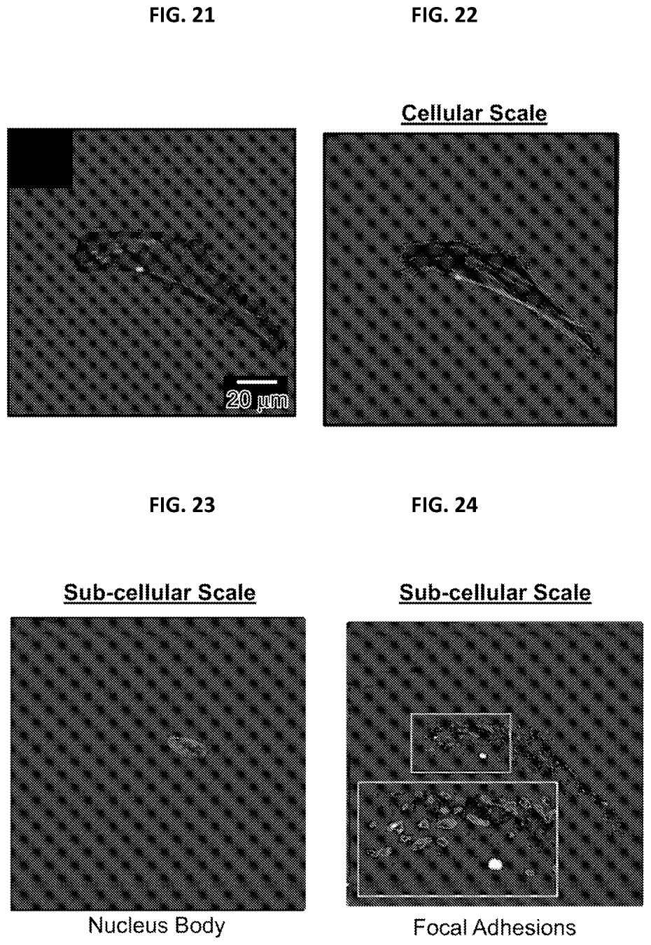

quantitative fluorescence confocal microscopy. Sample such images are shown in FIGS. 21-24, where a non-segmented view of a whole cell, a segmented view of just the cell body, a segmented view of just the nucleus body and a view displaying only the cell's focal adhesions are shown.

In some embodiments, a focal adhesion (FA) metrology framework allows

the definition of metrics that model the distribution of the FA proteins at the cell level. It can be understood as including three phases, as illustrated in FIG. 20.

First is the data acquisition phase, where the samples are images obtained with a high resolution confocal microscope equipped with 3 laser lines at 63× magnification and the samples are scanned across their thickness with a 0.1 μm step size. In this way, 3 sets of grayscale raw images can be produced for each cell, corresponding to the cellular and sub-cellular features of interest: e.g., FAs, Actin Microfilaments, and Nuclei, as depicted in FIGS. 21-24.

During the image processing phase, an algorithmic workflow where FAs can be automatically detected and segmented in each raw grayscale fluorescent image can be used, allowing for the 3-D volume reconstruction of all of the FAs within one cell in an xyz Cartesian coordinate system.

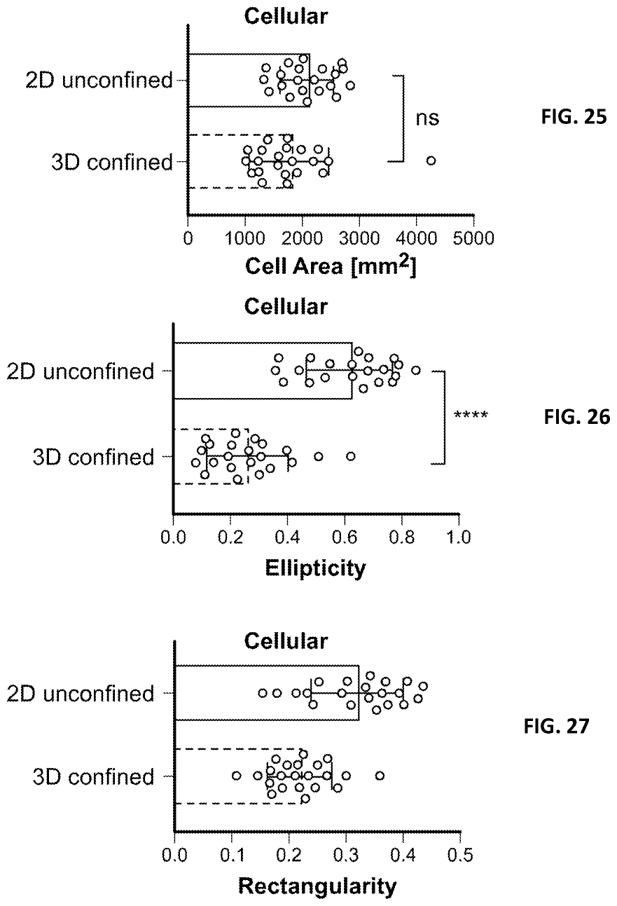

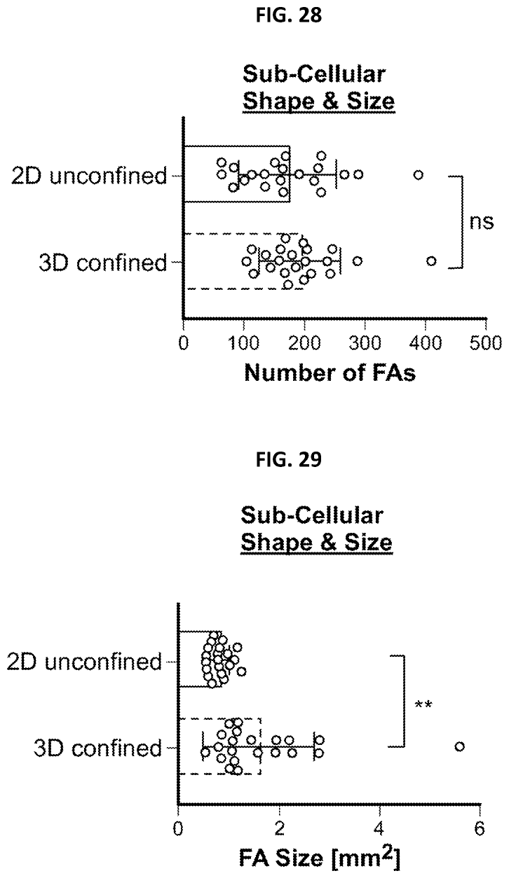

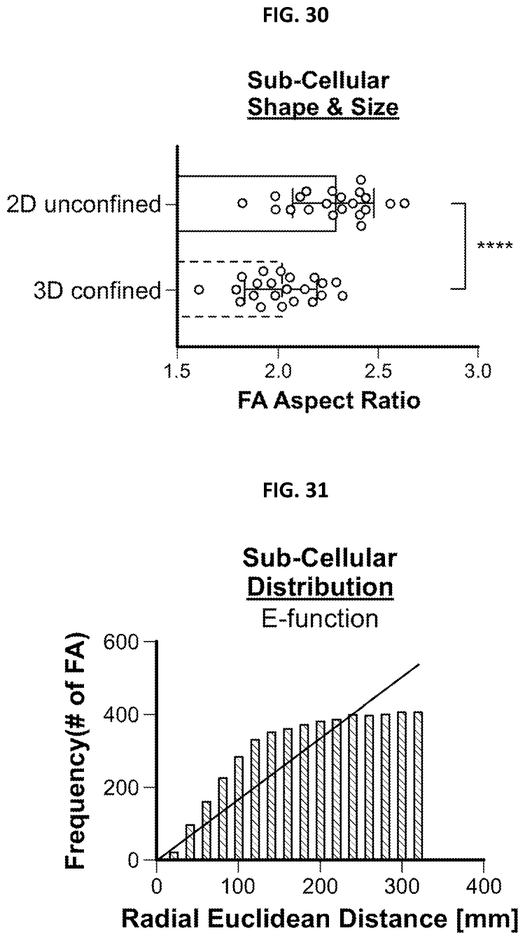

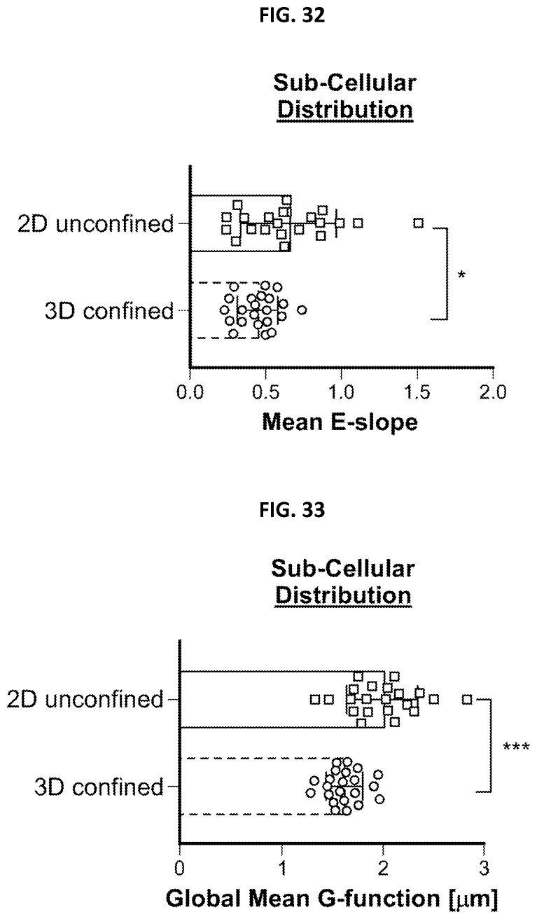

The image processing algorithmic procedure allows the development of critical cellular and subcellular focal adhesion morphometric and distribution metrics that are useful for the training and application of the developed classification method to various cell types according to embodiments of the present disclosure. The results are depicted in FIGS. 25-33. During the modeling phase, metrics that describe the distribution characteristics of the proteins can be defined. The values of these metrics could possibly be FA-representative of the whole cell population within each sample.

In embodiments, focal adhesions can be detected and segmented according to the algorithm. Initially, the cell body may be generated using thresholding and filtering techniques from a raw grayscale image colored green. Then, the individual FAs may be detected and accurately segmented within the detected region of interest. Specifically, “Clahe,” which stands for “contrast limited adaptive histogram equalization,” may be used to equalize image brightness and contrast across the processed image.

In some embodiments, a thresholding step may be performed, which automatically designates pixels as black or white based on whether they are above or below a certain pixel value.

In some embodiments, a dilation step may be performed, in which white pixels may be removed if they are surrounded by a number of black pixels greater than or equal to the specified value.

In some embodiments, an erosion step may be performed, in which black pixels may be removed in the same way as white pixels are removed in the dilation step.

In some embodiments, a reject features step may be performed in which infinite areas corresponding to white or black pixels may be removed.

In some embodiments, a Wiener filter may be applied, which reduces the sparse noise while preserving edges.

In some embodiments, a fast Fourier transform may be performed to reduce background noise and artifacts.

A manual review of the algorithm's output may be performed to verify the accuracy of the algorithm.

Following the same image processing algorithmic workflow for not only the FA channel, but also for the Actin Microfilament and DAPI channel allows for the 3-D volume reconstruction of each feature. They can then be merged into a composite image for visual inspection.

Two metrics were developed for the SIT algorithm. In particular, the radial Euclidean distances between focal adhesion and nuclei centroids were recorded for both a 2-D petri dish control and a 3-D confined and suspended state (i.e., 0-45° scaffold) system. Frequency distribution modeling was performed based on the Euclidean distance. A function was developed to characterize the relationship between radial Euclidean distances and the frequency of FAs within such a distance. From this E-function the slope was taken as the E-slope parameter. An increase in this E-slope parameter correlates with the formation of more FAs closer to the nucleus.

A similar frequency distribution modeling was also performed with the distance from each focal adhesion to its closest neighbor. A G-function was generated based on the relationship between nearest neighbor distance and the frequency of focal adhesions in this range. A smaller G-function value correlates with a more aggregated FA pattern at the individual/single cell level.

A morphometric analysis found that FA number and total area of FAs were not statistically significant when comparing melt electrospinning writing scaffolds with conventional controls (i.e., randomly electrospun meshes and a glass medium). However, FA size was higher for the MEW scaffold. Additionally the aspect ratio of the FAs in this experiment correlated with the ellipticity of the cell shape.

It was also found that cell area had no statistical differences between the four conditions, though the random fibrous substrates did have greater solidity. Without being bound by any particular theory, this is believed to be due to these fibers introducing random candidate cell attachment, resulting in more ruffled cell-shapes. Meanwhile 0-45° MEW-printed scaffolds saw triangular cell shapes with distinct cell attachment points. Thus, the MEW embodiment saw lower rectangularity and ellipticity.

A 7-D Cartesian coordinate system of cell shape phenotypes, in which each axis represents a feature metric, was developed for the 7 metrics computed from the Morphometric described above. a) Global (over a population of cells) E-slope (“I”), b) Rectangularity (“II”), c) Global (over a population of cells) mean G-function (“III”), d) FA size (“IV”), e) FA Aspect Ratio (“V”), f) Ellipticity (“VI”), and g) Cell Area (“VII”) were chosen as the seven dimensional parameters. Within this representation, each point represents one single cell feature-vector with 7 elements corresponding to the computed metrics for the specific cell. All metrics are normalized using a Z-score function, which centers and scales all metric values to have zero mean and unit standard deviation, respectively. The transformed metric vectors for each cell population are multidimensional datasets to train a Support Vector Machine (SVM) with a linear kernel using the classification learner package in MATLAB. The linear-kernel SVM is a supervised machine learning algorithm that can classify the data by finding the best hyperplane that separates all data points into: a) a class representing cells being in a 2-D unconfined state (Class A) and b) a class representing cells being in a 3-D confined state (class D). The best hyperplane for the SVM algorithm is considered the one with the largest margin between the two classes with the margin being the maximum width of the slab parallel to the hyperplane that has no interior data points.

The predictive accuracy of the linear-kernel SVM can be assessed using a 5-fold cross-validation scheme to protect against overfitting. Here, the data are randomly partitioned in 5 folds where, for each fold, the scheme trains the linear SVM using the out-of-fold observations and assesses the model performance using the in-fold data. The classification accuracy is defined as the average percentage of the correctly classified data for each fold and used as a metric for the classifier's predictive performance.

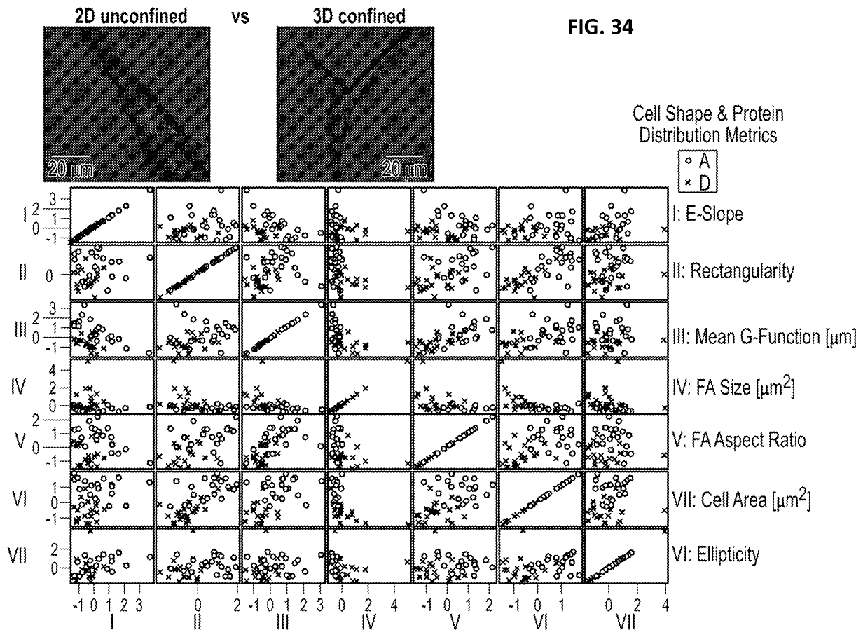

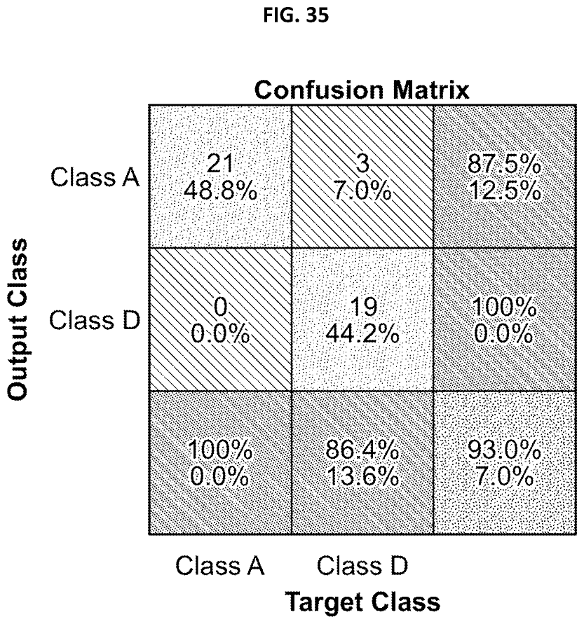

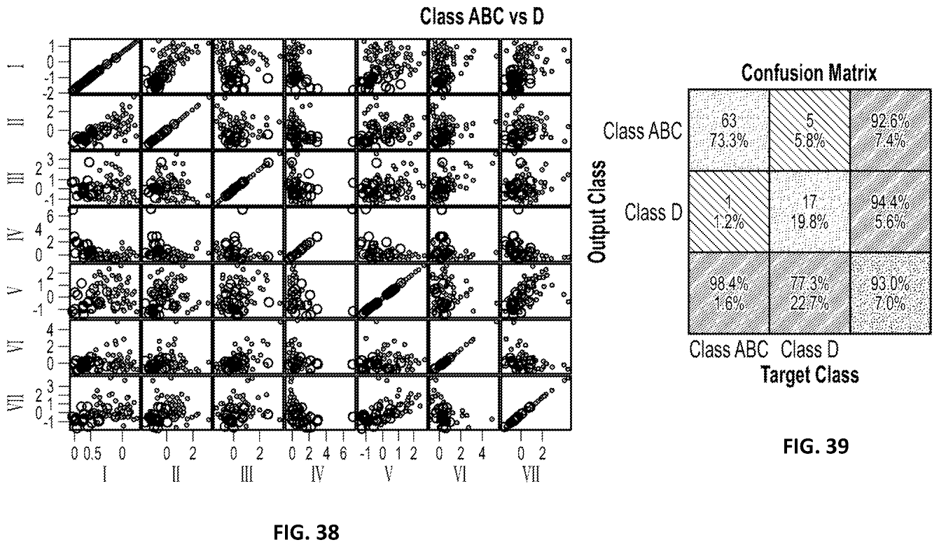

The results of the machine learning task, which is the classification of cell shape phenotypes modeled for every scaffold are depicted in FIGS. 34-39. While the initial assessment of the discriminatory information of each metric provides valuable insights concerning the cell shape phenotypic differences across and within each cell population group, the ability to infer the substrate dimensionality and architecture directly from single cell morphologies remains to be validated. To accomplish that, the single-cell multi-dimensional data sets are used to train the chosen machine learning algorithm with the aim of distinguishing between four different classes by considering all features simultaneously. The class declaration is depicted in Table 5 below, where all substrate dimensionalities and topographies are depicted along with the cell confinement states:

TABLE 5

| | Substrate | Cell |
|---|---|---|
| | Dimensionality - | Confinement State |
| Class | Architecture | (OBSERVATIONS) |
| A | 2-D uniform | unconfined |
| | (Controls - Glass surfaces) | |
| B | random | confined |
| | (SES - 1 min) | |
| C | random | confined |
| | (SES - 3 min) | |
| D | 3-D uniform | confined |
| | (MEW \| 0-45°) | |

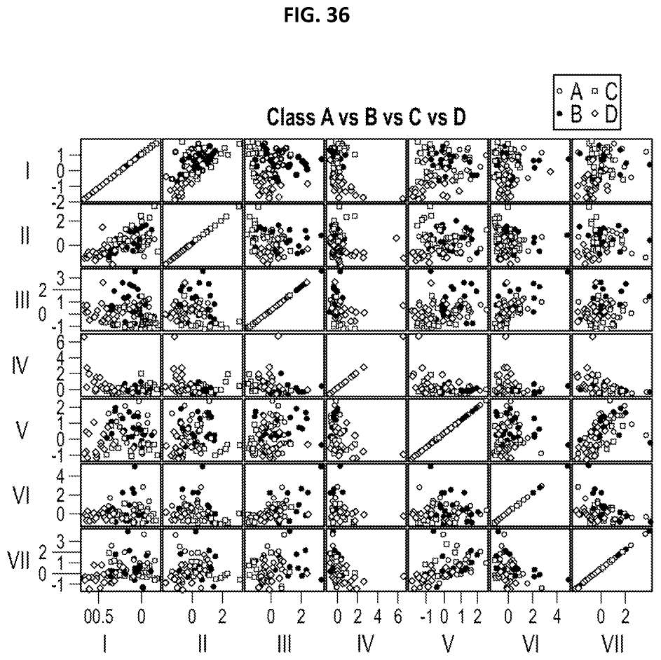

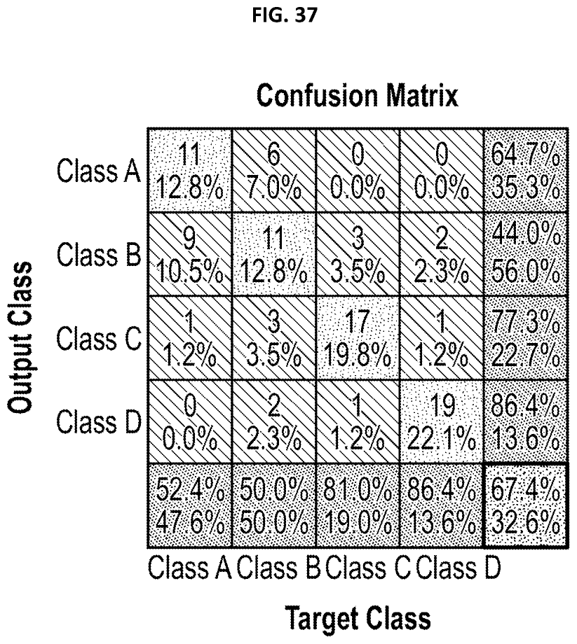

Three different classification tasks are performed. Combinations of the scaled metrics are plotted to allow easier assessment of the results (FIGS. 34-39). The capability of the classifier to operate satisfactorily with data outside the training set for each classification task is assessed based on the classification accuracy. Initially, the multi-class classification problem is attempted by taking into account cell morphologies across all the fabricated substrates (FIGS. 36 and 37). The classifier demonstrates a low classification accuracy (67%), which (without being bound by any particular theory) can be explained by the large intra-class variance of Class B. By removing Class B, the classification accuracy increases to 90.6%, demonstrating that the trained classifier can predict with high accuracy the substrate from which a cell originates based strictly on its feature vector identity. Remarkably, when the binary classification task is run by combining all classes corresponding to the flat or electrospun SES substrates, including the “noisy” Class B against Class D, the classification accuracy level remains around 93%. Thus, it is demonstrated that the 3-D microscale precision-stacked substrates promote a confined and suspended state that morphologically stands out both at the cellular as well as the sub-cellular FA level.

It is concluded that the MEW substrates may promote less migratory early cell shape phenotypic responses that are characteristic of a confined and suspended state. These responses are distinct from the confinement states adopted by the more actively motile cells on the flat and electrospun SES substrates. In the former case, cells tend to develop a more aggregated pattern of larger and less elongated mature FAs within cell bodies. The global shapes of the cells are dictated by the substrate's porous microarchitecture (e.g., triangular porous microarchitecture). In the latter case, cells tend to develop a more dispersed pattern of mature FAs within more elliptic cell bodies. Across the 2-D substrates, the degree of the resultant cell confinement appears to be regulated by the extent of fiber coverage with the cells on the control substrate (0% of fiber coverage) being in an unconfined state. Lastly, the substrates' structural heterogeneity with respect to fiber diameter and pore size distribution dictates the variance of the defined morphometric and protein distribution metrics with the MEW I 0-45° and SES-3 min substrate demonstrating the most and least homogeneous population of single cell morphologies, respectively.

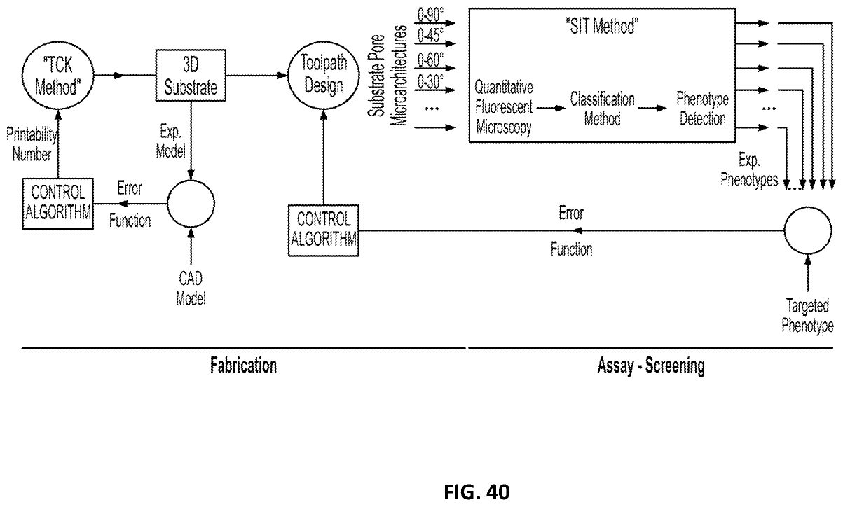

Integration of embodiments of the TCK fabrication method and embodiments of the SIT classification scheme enable discovery of the extent and time duration over which stem cells conserve their shapes and phenotypes, thereby facilitating manipulation of the shapes and phenotypes of the stem cells using the geometry of the scaffold or the bioreactor substrate as a tool. A schematic diagram of a concept for industrial exploitation of the classification method according to embodiments of the present disclosure, further including feedback and feedforward control methodologies for the programmable expansion and harvesting of stem cells having phenotypes that are targeted and realized according to a method of the present disclosure is depicted in FIG. 40. By such means, stem cell therapies can be improved significantly by tailoring the geometries of scaffolds and bioreactors used during the administration of such therapies.

Using fibroblasts as a model cell system or stem cell surrogate, the mechano-sensing response of adherent cells is investigated as a function of variable substrate dimensionality (2D vs. 3D) and porous geometry (or microarchitecture) (randomly oriented, “non-woven” vs. precision-stacked, “woven”). Single-cell confinement states are modeled using confocal fluorescence microscopy in conjunction with an automated single-cell bioimage data analysis workflow that extracts quantitative metrics of the whole cell and sub-cellular focal adhesion protein features. The extracted multi-dimensional data set is employed to train a machine learning algorithm to classify cell shape phenotypes, as discussed above and in paragraphs 0088 et seq. of U.S. patent application Ser. No. 15/998,685, titled “Integrated methods for precision manufacturing of tissue engineering scaffolds,” to Tourlomousis et al. (assigned to the same Assignee as the present application), the entire content of which is incorporated herein by reference.

Cells acquire shapes under culture that are directly related to the geometry (or architecture) of the lattice sub-structure on which they are attached. And cells assume distinct confinement states that are enforced by the prescribed lattice sub-structure dimensionalities and porous geometries (or microarchitectures) with the woven MEW substrates promoting the highest cell shape homogeneity compared to non-woven fibrous substrates. In contrast to the diversity in cell shapes observed when cells are cultured on nonwoven, randomly structured substrates with similar filament diameters, a high degree of cell shape uniformity can be achieved with the lattice sub-structures constructed of filaments with diameters as small as 10 micrometers (or having a filament diameter of about 10 to about 100 micrometers) according to embodiments of the present disclosure.

Immunofluorescence imaging may be used to generate positive and negative cell surface marker expressions to characterize the phenotype, or observable biological outcome, of mesenchymal stem cells. This immunofluorescence imaging indicates that mesenchymal stem cells lose their characteristic phenotype and differentiate within one week of culturing when lattice sub-structures with random, “uncontrolled,” mesh structures are used. Such a rapid loss of the phenotype would decrease the expansion potential of the MSCs and introduce problems with control of the purity and homogeneity of the MSC population. The immunofluorescent positive and negative surface markers also demonstrate that mesenchymal stem cells cultured on porous meshes (i.e., lattice sub-structures) with uniform lattice structures constructed out of fine filaments (i.e., diameters of around 10 μm) proliferate without differentiation (i.e., “conserved their stemness”) for durations that are significantly longer than those that could be achieved on substrates with random non-woven geometries. The immunofluorescent imaging also confirms that the type of lattice geometry affects the cell shape and functional “phenotype.”

These procedures used to generate the database also confirm the achievability of populations of cells with uniform cell shapes. This could be useful in biomedical research and cell based therapies since cell shape governs cell function, and the lattice sub-structures, biomaterial structures and methods according to embodiments of the present disclosure can be used to engineer and quantify cell responses with unprecedented precision and reproducibility.

According to embodiments of the present disclosure, this database of lattice sub-structures (with myriad lattice geometries) and their corresponding time-dependent changes in the phenotypes of seeded stem cells can be used to design and construct functionally graded biomaterial structures (e.g., biomaterial substrate modules 100 or multi-module biomaterial substrates 200). Functionally graded biomaterial structures can better mimic certain important gradients observed in native tissues.

Indeed, using the correlated information in the database (i.e., the data correlating a particular lattice sub-structure with a particular single cell shape and a particular long-term tissue formation), a relevant biomaterial structure can be designed based on the desired tissue formation. For example, in some embodiments, a biomaterial substrate module 100 can be designed and manufactured. In some embodiments, for example, the correlated information in the database can be used to determine what combination of various lattice sub-structures are needed to generate the desired tissue or organ type. In some embodiments, at least two of the lattice sub-structures in the designed combination have a different lattice geometry. As used herein, the expression “different lattice geometry” refers to the two lattices (or lattice sub-structures) having at least one different geometrical parameter (e.g., a different inter-layer angle, a different fiber diameter, or a different inter-fiber spacing). It is understood, therefore, that two lattices (or lattice sub-structures) may have one or more geometrical parameters that are the same, but still have different lattice geometries (so long as at least one parameter is different). As an example, as shown in FIG. 2A, in some lattice sub-structure combinations, two or more of the lattice sub-structures (denoted architectures #1-3 or A1, A2 and A3 in the drawing) may have the same inter-layer angle, but different fiber diameter and/or inter-fiber spacing. This can lead to a tissue- and/or organ-scale biomaterial substrate module 100 with a structural gradient (shown by the arrows in FIG. 2B (where structural stiffness increases (and thus, porosity decreases) from top to bottom), 2C (where structural stiffness increases (and thus, porosity decreases) from left to right) and 2D (where structural stiffness increases (and thus, porosity decreases) from front to back)) that gives rise to multiple tissue types, as shown, for example, in FIGS. 2B through 2D. Specifically, the different structural stiffnesses (or porosities) of the component lattice substructures (or regions thereof) result in different cell phenotypes, giving rise to multiple tissue types grown or formed on the resulting biomaterial substrate module 100. For example, the structural gradient shown by the arrows in FIG. 2B is indicative of the targeted control of cell shape and resultant tissue type that change from top to bottom, and the structural gradient in FIG. 2C is indicative of the targeted control of cell shape and resultant tissue type that change from left to right. And the structural gradient in FIG. 2D is indicative of the targeted control of cell shape and resultant tissue type that change from front to back.

As used herein, the term “structural stiffness” denotes the physical stiffness of the biomaterial structure imparted by the geometrical features of the biomaterial structure and the arrangement of filaments in the biomaterial structure, and does not refer to the inherent stiffness of the material making up the filaments. Indeed, the geometrical features of the biomaterial structures (e.g., filament diameter, inter-filament distance and filament orientation) define a porosity of the biomaterial structure, and that porosity affects the structural stiffness of the biomaterial structure. Specifically, as porosity increases, structural stiffness decreases, and vice versa.

The correlated information in the database may also (or alternatively) be used to design and manufacture a multi-module biomaterial substrate 200. It is understood that the design and manufacture of the multi-module biomaterial substrate 200 does not require the prior design and manufacture of a biomaterial substrate module or modules 100, and that the multi-module biomaterial substrate 200 may be designed directly, without an intermediate module 100 design. Additionally, while the homogenous and heterogeneous biomaterial substrate modules 100 are constructed from combinations of lattice sub-structures 110, the multi-module biomaterial substrates 200 may be constructed from combinations of biomaterial substrate modules 100 (e.g., modules 100a through 100d in FIG. 3B), or from combinations of biomaterial substrate modules 100 and individual lattice sub-structures 110. For example, as discussed generally above, the multi-module biomaterial substrates 200 may include: a plurality of the same or different heterogeneous biomaterial substrate modules; a plurality of homogeneous biomaterial substrate modules (at least two of which are different from each other); a combination of heterogeneous biomaterial substrate modules and homogenous biomaterial substrate modules; a combination of heterogeneous biomaterial substrate modules and individual lattice sub-structures; a combination of homogeneous biomaterial substrate modules and individual lattice sub-structures; or a combination of heterogeneous biomaterial substrate modules, homogeneous biomaterial substrate modules, and individual lattice sub-structures. In this way, the multi-module biomaterial substrates 200 can have a wide variety of graded or otherwise differing geometries throughout the structure, giving rise to a wide variety of tissue types and tissue formations (including, e.g., organ structures). For example, as shown generally in FIG. 3A, in some embodiments, multi-module biomaterial substrates 200 may be designed and fabricated by combining various biomaterial substrate modules 100 and/or other lattice sub-structures 100 that are known from the database to induce specific representative single cell shapes and specific long-term tissue types in the long term. As shown generally in FIG. 3B, this can lead to a tissue- and/or organ-scale biomaterial structure with both a structural gradient (as indicated by the arrow associated with the biomaterial substrate module 100a, with structural stiffness increasing (and thus, porosity decreasing) within that module from left to right) and different lattice geometry types, which can give rise to multiple tissue types. Accordingly, employing the correlations in the database, a wide variety of biomaterial structures can be designed and fabricated based on the desired tissue type or formation, and this can be accomplished in a programmable way.

As a further illustration of the tissue capabilities of the biomaterial structures according to embodiments of the present disclosure, FIG. 4 illustrates the use of two different lattice sub-structures to create a heterogeneous biomaterial substrate module. This heterogeneous module is used to form an “interface tissue,” including a softer tissue on one side and a harder tissue on the other side. As shown, the overall structure of the heterogeneous module includes two distinct lattice sub-structures having different lattice geometries. As also shown, the depicted heterogeneous module is designed to create a specific osteo-chondral interface tissue having chondrocytes on one side and osteoblasts on the other.

Indeed, seeding of stem cells on the surfaces 5 and 6 of the two distinct lattice sub-structures of the heterogeneous module could lead to differences in cell shapes within 24 hours, and different cell phenotypes upon culturing until confluence. In this example, the tissue construct generated by the geometry of the first lattice sub-structure (denoted 5 in FIG. 4) could guide the differentiation of the stem cells towards “chondrocytic differentiation,” while the tissue construct generated by the geometry of the second lattice sub-structure (denoted 6 in FIG. 4) could guide the differentiation of the stem cells towards “osteoblastic differentiation.” Upon culturing, the tissue construct could become the “interface tissue,” with one side (i.e., 5) having soft tissue (e.g., cartilage) and the other side (i.e., 6) having hard tissue (e.g., cancellous or cortical bone).

As can be seen from this illustration, systematic changes in the types of lattice geometries used to construct the biomaterial structures can lead to a series of cell shapes upon seeding, and to tissues with “phenotype” gradients, enabling the construction of complex tissue formations (including, e.g., organ structures). For example, a biomaterial structure according to embodiments of the present disclosure could be constructed using the information from the database to produce a tissue formation that mimics the complex native tissue formation shown in FIG. 5. According to some embodiments, the database may be used to construct a digital model (or map) of the multi-module biomaterial substrate needed to construct the mimicking tissue formation. For example, such a biomaterial structure might include an inner lattice sub-structure (or biomaterial substrate module) having a lattice geometry that can lead to the differentiation of stem cells into endothelial cells. This inner lattice sub-structure (or biomaterial substrate module) could enable the formation of an endothelium (i.e., the linings of blood vessels when lumen-like structures are generated). And additional lattice sub-structures (or biomaterial substrate modules) could be assembled with the inner (endothelial) lattice sub-structure and designed to lead to the differentiation of the stem cells into different phenotypes (e.g., those needed for the formation of the muscle and elastic membranes). As can be seen from this illustration, the structures and methods according to embodiments of this disclosure can be used to form complex tissues and tissue formations (including, e.g., organ structures) by systematically varying the lattice geometries within the biomaterial structures to give rise to the desired cell phenotypes.

This ability to design cell-instructive geometrical/biophysical cues across the length, width and height of 3D biomaterial structures, as described herein, can be used in a wide variety of fields, including, but not limited to the development of novel cell expansion platforms for cell-based therapies, organ-on-a-chip platforms for screening drug/vaccine toxicity and efficacy, in vitro disease/physiological models, acellular biomaterial structures that can be used as implants for guiding healthy tissue formation within lesions, and the bioengineering of tissue bulk/tissue interface and/or organ analogs. Indeed, the ability to better control the differentiation of stem cells into specific cell types enables the production of differentiated cells that can possibly be used in research aimed at cell-based therapies to treat, for example, heart disease, diabetes, vision and hearing loss, traumatic spinal cord injury, Duchenne's muscular dystrophy, etc.

Further tailoring of the mechanical properties and/or cellular response of the biomaterial structures can also be accomplished by the addition of fillers and/or additives, similar to the creation of conventional stiffness gradients (described in general above). Any suitable such fillers and/or additives may be used for this purpose without limitation, and the incorporation of these fillers and/or additives is described in detail in Erisken, et al., “Functionally graded electrospun polycaprolactone and β-tricalcium phosphate nanocomposites for tissue engineering applications,” Biomaterials, vol. 29, pgs. 4065-4073 (2008), the entire content of which is incorporated herein by reference. For example, as noted in Erisken, et al., “Functionally graded electrospun polycaprolactone and β-tricalcium phosphate nanocomposites for tissue engineering applications,” Biomaterials, vol. 29, pgs. 4065-4073 (2008), the entire content of which is incorporated herein by reference, nonlimiting examples of the filler/additive may include β-tricalcium phosphate, hydroxyapatite, calcium carbonate, carbon nanotubes, hydrogels, proteins, collagen, polyglycolic acid, poly-lactic-co-glycolic acid (PLGA), hyaluronan, calcium phosphate, fibrin, bioactive glass, etc. In some embodiments, the filler/additive includes β-tricalcium phosphate.

The concentration of the filler/additive is also not particularly limited, and may be any concentration suitable to achieve the desired mechanical properties and cellular response when considered together the structural gradient described above. However, in some embodiments, as noted in Erisken, et al., “Functionally graded electrospun polycaprolactone and β-tricalcium phosphate nanocomposites for tissue engineering applications,” Biomaterials, vol. 29, pgs. 4065-4073 (2008), the entire content of which is incorporated herein by reference, the filler/additive may be provided in a concentration of about 0 to about 15% by weight. Additionally, the filler/additive may be present in a concentration gradient in which the concentration of the filler/additive varies across the biomaterial structure. The concentration of the filler/additive at any point along the gradient may also vary from about 0 to about 15% by weight, but the present disclosure is not limited thereto.

Moreover, because the filler/additive also contributes to the cellular response of the biomaterial structure, according to embodiments of the present disclosure, the database may also store filler/additive material and concentration (or concentration gradient) data. The database may correlate this filler/additive and concentration data with the data described above, i.e., correlating predicted cell differentiation types or predicted long term tissue structures with lattice sub-structures having specified geometric parameters, to produce a digital model or instructions for a combination of the biomaterial structure and filler/additive material and concentration (or concentration gradient) needed to form the desired tissue type or organ structure.

Additionally, the functionality induced by tailoring different geometries of the biomaterial structures (e.g., lattice sub-structures, biomaterial substrate modules, and/or multi-module biomaterial substrates), as described herein, can be extended from programmable cellular responses acquired by the biomaterial structures seeded with cells in a laboratory setting (in vitro) to programmable cellular responses in a host organism (in vivo), which may be animal or human. These in vivo cellular responses can be achieved, for example, by implantation of a designed acellular biomaterial structure into the host organism. As described herein, the acellular biomaterial structure may be designed (or tailored) to guide the growth of healthy tissue surrounding the biomaterial structure after implantation. This design (or tailoring) may be accomplished, as described herein, by tailoring the lattice geometry types to grow the target tissue in vivo (i.e., the tissue in which the biomaterial structure is implanted) in the same way the lattice geometry types are designed (or tailored) for growth of tissue on the biomaterial structures in vitro.

The ability to intelligently tailor cell-instructive geometrical/biophysical cues of the biomaterial structures also enables the design of implantable biomaterial structures that minimize or prevent the host organism's “foreign body response,” which typically encapsulates the implanted medical device in a fibrotic membrane. This “foreign body response” is known to those of ordinary skill in the art, and is described generally in Grainger, “All charged up about implanted biomaterials,” Nature Biotechnology, vol. 31, no. 6, pgs. 507-509, the entire content of which is incorporated herein by reference. However, mechanical matching of the implant to the host tissue can minimize or prevent the foreign body response, as discussed in Carnicer-Lombarte, et al., “Mechanical matching of implant to host minimises foreign body reaction,”https://doi.org/10.1101/829648 (pre-print publication), the entire content of which is incorporated herein by reference. Accordingly, in some embodiments of the present disclosure, the geometries and porous microarchitectures of the biomaterial structures can be adjusted/tailored to prevent or minimize the foreign body response. For example, the geometries and porous microarchitecture can be designed not only to grow the specified target tissue in vivo, but can also be tailored to match the mechanical or elastic modulus (and/or other mechanical properties, e.g., surface stiffness) of the target tissue.

Loss of mass after implantation is another challenge of designing effective biomaterial implants, as described in Hutmacher, “Scaffolds in tissue engineering bone and cartilage,” Biomaterials, vol. 21, pgs. 2529-2543 (2000). However, matching of the mechanical properties (e.g., elastic modulus) provides the biomaterial structure with sufficient initial mechanical strength to substitute for the missing or damaged target tissue in the host organism while also allowing for a degradation rate of the implant that is compatible with tissue growth and maturation rate of the target tissue. See, e.g., Woodruff, et al., “Bone tissue engineering: from bench to bedside,” Materials Today, vol. 15, no. 10, pgs. 430-435, October 2012, the entire content of which is incorporated herein by reference.

In some embodiments, the mechanical properties (e.g., elastic modulus) of the biomaterial structure may be tailored to match those of the target tissue (and thereby minimize or prevent the foreign body response) by adjusting the geometry (or structural gradients) of the biomaterial structures alone. According to some embodiments, though, the biomaterial structure may also be coated in a material that has a mechanical or elastic modulus that matches that of the target tissue. These coating materials and techniques are also known to those of ordinary skill in the art, and any suitable such material and technique may be used for this purpose without limitation. For example, those materials and techniques disclosed in Carnicer-Lombarte, et al., “Mechanical matching of implant to host minimises foreign body reaction,” https://doi.org/10.1101/829648 (pre-print publication), may be used. Some non-limiting examples of such suitable coating materials include silicones, polymers (e.g., polyacrylamides), hydrogels, collagen, etc. The thickness of the coating is not particularly limited, but should be thick enough to effectively match the surface mechanical properties of the implant (i.e., the biomaterial structure) to those of the target tissue. In some embodiments, for example, the thickness of the coating may be about 50 microns to about 150 microns, e.g., about 100 microns.

While certain exemplary embodiments of the present disclosure have been illustrated and described, those of ordinary skill in the art will recognize that various changes and modifications can be made to the described embodiments without departing from the spirit and scope of the present disclosure, and equivalents thereof, as defined in the claims that follow this description. For example, although certain components may have been described in the singular, i.e., “a” lattice sub-structure, “a” biomaterial substrate module, and the like, one or more of these components in any combination can be used according to the present disclosure.

Also, although certain embodiments have been described as “comprising” or “including” the specified components, embodiments “consisting essentially of” or “consisting of” the listed components are also within the scope of this disclosure. For example, while embodiments of the present invention are described as comprising a combination of lattice sub-structures or a combination of biomaterial substrate modules, embodiments consisting essentially of or consisting of these components are also within the scope of this disclosure. For example, a biomaterial substrate module may consist essentially of a plurality of lattice sub-structures. In this context, “consisting essentially of” means that any additional components will not materially affect the chemical, biochemical, physical, biophysical or biophysiological properties of the biomaterial substrate module.

As used herein, unless otherwise expressly specified, all numbers such as those expressing values, ranges, amounts or percentages may be read as if prefaced by the word “about,” even if the term does not expressly appear. Further, the word “about” is used as a term of approximation, and not as a term of degree, and reflects the penumbra of variation associated with measurement, significant figures, and interchangeability, all as understood by a person having ordinary skill in the art to which this disclosure pertains. Any numerical range recited herein is intended to include all sub-ranges subsumed therein. Plural encompasses singular and vice versa. When ranges are given, any endpoints of those ranges and/or numbers within those ranges can be combined within the scope of the present disclosure. The terms “including” and like terms mean “including but not limited to,” unless specified to the contrary.

Notwithstanding that the numerical ranges and parameters set forth herein may be approximations, numerical values set forth in the Examples are reported as precisely as is practical. Any numerical value, however, inherently contains certain errors necessarily resulting from the standard variation found in their respective testing measurements. The word “comprising” and variations thereof as used in this description and in the claims do not limit the disclosure to exclude any variants or additions.

1. A biomaterial structure for proliferation, differentiation and/or expansion of stem cells, the biomaterial structure comprising at least one lattice sub-structure, the biomaterial structure having a structural gradient in which one or more geometrical features of the biomaterial structure varies along at least one dimension of the biomaterial structure in three-dimensional space.

2. The biomaterial structure of claim 1, wherein the at least one lattice sub-structure comprises at least first and second lattice substructures.

3. The biomaterial structure of claim 2, wherein the structural gradient of the biomaterial structure is accomplished by the first and second lattice sub structures having at least one different geometrical parameter.

4. The biomaterial structure of claim 1, wherein the at least one lattice sub-structure, the first and second lattice sub-structures, or the biomaterial structure are constructed of filaments having a diameter of about 10 micrometers to about 100 micrometers.

5. The biomaterial structure of claim 1, wherein the at least one lattice sub-structure, the at least first and second lattice sub-structures, or the biomaterial structure comprises:
a first plurality of lattice sub-structures assembled into a first biomaterial substrate module; and
a second plurality of lattice sub-structures assembled into a second biomaterial substrate module,
the first and second biomaterial substrate modules assembled into a multi-module biomaterial substrate.

6. The biomaterial structure of claim 5, wherein at least two of the first plurality of lattice sub-structures of the first biomaterial substrate module have at least one different geometrical parameter.

7. The biomaterial structure of claim 5, wherein each of the second plurality of lattice sub-structures of the second biomaterial substrate module has identical geometrical parameters.

8. The biomaterial structure of claim 5, wherein:
each of the first plurality of lattice sub-structures of the first biomaterial substrate module has identical geometrical parameters;
each of the second plurality of lattice sub-structure of the second biomaterial substrate module has identical geometrical parameters; and
the first plurality of lattice sub-structures of the first biomaterial substrate module and the second plurality of lattice sub-structures of the second biomaterial substrate module have at least one different geometrical parameter.

9. The biomaterial structure of claim 1, wherein the at least one lattice substructure, the at least first and second lattice sub-structures, or the biomaterial structure comprises:
a first plurality of lattice sub-structures assembled into a first biomaterial substrate module; and
the second lattice sub-structure,
the first biomaterial substrate module and the second lattice sub structure assembled into a multi-module biomaterial substrate.

10. The biomaterial structure of claim 9, wherein at least two of the first plurality of lattice sub-structures of the first biomaterial substrate module have at least one different geometrical parameter.

11. The biomaterial structure of claim 9, wherein each of the first plurality of lattice sub-structures of the first biomaterial substrate module has identical geometrical parameters.

12. A method of making a biomaterial structure designed to grow a specified tissue formation or organ structure mimicking a native tissue formation or native organ structure, the method comprising:
generating a digital model of the biomaterial structure from a database correlating predicted cell differentiation types or predicted long term tissue structures with lattice sub-structures having specified geometric parameters, the digital model comprising:
at least one lattice sub-structure identified having a structural gradient identified by the database as needed to form each tissue type needed to mimic the native tissue formation or native organ structure, the structural gradient being such that one or more geometrical features of the biomaterial structure varies along at least one dimension of the biomaterial structure in three-dimensional space; and/or
a combination of lattice sub-structures identified by the database as needed to form each tissue type needed to mimic the native tissue formation or native organ structure, and
constructing or printing the biomaterial structure using the digital model.

13. The method of claim 12, wherein the digital model comprises the at least one lattice sub-structure having the structural gradient, a combination of different lattice sub-structures, a combination of different biomaterial substrate modules, or a combination of at least one lattice sub-structure and at least one biomaterial substrate module.

14. The method of claim 12, wherein the constructing or printing the biomaterial structure using the digital model comprises 3D printing the biomaterial structure using the digital model as an instruction or template.

15. The method of claim 12, wherein the constructing or printing the biomaterial structure using the digital model comprises manual connecting or assembling the biomaterial structure using the digital model as an instruction or template.

16. A method of tuning early single cell shape on a biomaterial structure, the method comprising varying the physical characteristics of the biomaterial structure in order to guide long term tissue function, wherein early single cell shape is the single cell shape formed 24 hrs after the cell has been seeded.

17. The method of claim 16, wherein the varying the physical characteristics of the biomaterial structure comprises imparting the biomaterial structure with a structural gradient in which one or more geometrical features of the biomaterial structure varies along at least one dimension of the biomaterial structure in three-dimensional space.

18. The method of claim 16, wherein the biomaterial structure comprises at least one lattice sub-structure, and the varying the physical characteristics of the biomaterial structure comprises imparting the at least one lattice sub-structure with a structural gradient in which one or more geometrical features of the lattice substructure varies along at least one dimension of in three-dimensional space.

19. The method of claim 16, wherein the at least one lattice sub-structure or the biomaterial structure comprises at least first and second lattice substructures.

20. The method of claim 19, wherein the structural gradient of the biomaterial structure is accomplished by the first and second lattice sub-structures having at least one different geometrical parameter.

21. The method of claim 16, wherein the at least one lattice sub-structure, the first and second lattice sub-structures or the biomaterial structure are constructed of filaments having a diameter of about 10 micrometers to about 100 micrometers.

22. The method of claim 16, wherein the at least one lattice sub-structure, the at least first and second lattice sub-structures, or the biomaterial structure comprises:
a first plurality of lattice sub-structures assembled into a first biomaterial substrate module; and
a second plurality of lattice sub-structures assembled into a second biomaterial substrate module, the first and second biomaterial substrate modules assembled into a multi-module biomaterial substrate.

23. The method of claim 22, wherein at least two of the first plurality of lattice sub-structures of the first biomaterial substrate module have at least one different geometrical parameter.

24. The method of claim 22, wherein each of the second plurality of lattice sub-structures of the second biomaterial substrate module has identical geometrical parameters.

25. The method of claim 22, wherein:
each of the first plurality of lattice sub-structures of the first biomaterial substrate module has identical geometrical parameters;
each of the second plurality of lattice sub-structure of the second biomaterial substrate module has identical geometrical parameters; and
the first plurality of lattice sub-structures of the first biomaterial substrate module and the second plurality of lattice sub-structures of the second biomaterial substrate module have at least one different geometrical parameter.

26. The method of claim 16, wherein each of the at least one lattice substructure or the at least first and second lattice sub-structures comprises:
a first plurality of lattice sub-structures assembled into a first biomaterial substrate module; and
the second lattice sub-structure,
the first biomaterial substrate module and the second lattice sub structure assembled into a multi-module biomaterial substrate.

27. The method of claim 26, wherein at least two of the first plurality of lattice sub-structures of the first biomaterial substrate module have at least one different geometrical parameter.

28. The method of claim 26, wherein each of the first plurality of lattice sub structures of the first biomaterial substrate module has identical geometrical parameters.
